(19) United States
(12) Patent Application Publication
(10) Pub. No.: US 2019/0046481 A1
(43) Pub. Date: Feb. 14, 2019

(54) TRANSDERMAL FORMULATIONS FOR DELIVERY OF PROPIONIC AND ACETIC ACID NSAIDS, AND THEIR USE IN THE TREATMENT OF NSAID-RESPONSIVE DISEASES AND CONDITIONS

(71) Applicant: DELIVRA INC., Hamilton (CA)

(72) Inventors: Joseph Gabriele, Stoney Creek (CA); Mikaela Teris, Montreal (CA)

(21) Appl. No.: 15/759,644

(22) PCT Filed: Sep. 19, 2016
(86) PCT No.: PCT/CA2016/051095

§ 371 Date: Mar. 13, 2018

Related U.S. Application Data
(60) Provisional application No. 62/220,605, filed on Sep. 18, 2015.

(51) Int. Cl. A61K 31/192; A61K 9/00; A61K 9/06; A61K 47/24; A61P 29/00; A61P 35/00; A61P 19/02

(52) U.S. Cl. CPC A61K 31/192; A61K 9/0014; A61K 9/06; A61K 9/0002; A61K 31/196; A61P 29/00; A61P 35/00; A61P 19/02; A61K 2300/00; A61K 47/24

(57) ABSTRACT

The present application is directed to transdermal formulations for the delivery of propionic and acetic acid NSAIDs to a subject for the treatment of NSAID-responsive diseases. In particular, the transdermal formulation is an emulsion comprising an oil phase, an aqueous phase and an external phase.

### CROSS-REFERENCE TO RELATED APPLICATIONS

This application claims the benefit of U.S. Provisional Patent Application Ser. No. 62/220,605, filed Sep. 18, 2015, the content of which is hereby incorporated by reference in its entirety.

### FIELD

The present application relates to transdermal formulations for effective delivery of propionic and acetic acid NSAIDs and various methods of use thereof.

### BACKGROUND

Non-Steroidal Anti-Inflammatory Drugs (NSAIDs) are the largest group of non-narcotic analgesics, and are categorized chemically into six distinct classes including, the anthranilic acid derivatives, enolic acid derivatives, pyrazole derivatives, salicylates, propionic and acetic acid derivatives. Common to all these drugs is their inhibition of prostaglandin synthesis, which contributes to their favorable analgesic and pharmacological properties as well as to their principal side effect, gastrointestinal irritation (Brogdan R. N, 1986).

Generally, NSAIDs are relatively lipid-soluble and are weak acids having a pKa in the range of 3 to 5, which results in their favorable absorption profiles in the stomach and intestinal mucosa (Conaghan, 2012). However the rate of absorption varies between the classes of NSAIDs, which can impact their suitability for particular treatments and conditions. NSAIDs can also be categorized by their half-life into two groups: those with a short half-life (<6 h) and those with a long half-life. Proceeding oral administration of these drugs, their peak plasma concentrations are attained within 1 to 2 hours and they are rapidly bio-transformed with a serum half-life of 1.8 to 2 hours. The elimination half-lives (t_{1/2}) in most instances are in the range of 2-5 hours, although notable exceptions exist providing for very long half-lives of approximately 20-60 hours. Generally, most NSAIDs, like propionic and acetic acids, are completely eliminated in 24 hours through metabolism (Aslam et al., 2010).

Biological Targets of NSAIDs

NSAIDs provide treatment for acute or chronic conditions where pain and inflammation are present. They play an important role in the pain management of various clinical conditions including headaches, postoperative pain, musculoskeletal and soft tissue pain, rheumatoid arthritis, and osteoarthritis by blocking the conversion of arachidonic acid to cyclic endoperoxides by cyclooxygenase (COX) enzymes controlling the production of prostanoids (prostaglandins and thromboxane) (Anand et al., 1999). Generally, the COX enzyme catalyzes the formation of prostaglandins, which are known to act as messenger molecules in the process of inflammation. Two specific COX isoenzymes, COX-1 and COX-2 play a significant role, wherein COX-1 mediates the mucosal protection of gastrointestinal (GI) mucosa, while COX-2 is found throughout the body, including joints and muscle, and mediates effects on pain and inflammation (Canadian agency for drugs and technologies in health, 2013).

Most NSAIDs act as nonselective inhibitors of both the COX-1 and COX-2 isoenzymes, wherein the inhibition is competitively reversible. NSAIDs that show a higher selectivity for the COX-2 isoenzyme are favored, as the nonselective inhibitors can have adverse effects of GI bleeding. However, inhibition of COX, whether it is selective or nonselective, is the primary mechanism for NSAIDs and appears to provide the majority of the effect in decreasing pain and inflammation (Botting et al., 2006), although inhibition of COX-2 mainly leads to the anti-inflammatory, analgesic and antipyretic effects observed.

Due to the significant adverse effects through oral administration, including local or systemic disturbance in the GI tract, cardiovascular risk, low oral bioavailability due to extensive first pass metabolism and low enterohepatic circulation, the use of NSAIDs in clinical applications have been limited (Longping et al., 2007).

The premature metabolism of drugs as a result of the first-pass effect has made transdermal delivery an attractive and alternative strategy (Prausnitz, et al. 2008). Topical administration circumvents issues of low bioavailability and additionally results in fewer adverse effects and lower systemic drug concentration.

For many years, people have placed natural substances on the skin for local ailments. However, the human skin acts as a formidable barrier due in large part to the stratum corneum, which mostly consists of a lipid-enriched matrix and blocks entry of most topically applied agents, with the exception of low molecular weight, lipid-soluble drugs. This poses a challenge for administrating medications via the skin for either local cutaneous or systemic therapy.

Transdermal drug delivery strategies have thus focused primarily on the manipulation of the lipid milieu of the skin. In particular, penetration enhancers which interact with skin constituents to promote drug transport have provided an approach to increase the range of therapeutic agents that can be delivered transdermally.

Despite the significant permeability barrier of the stratum corneum, drug delivery via the skin is a very attractive option and is widely employed for both local and systemic therapy. Topical treatment of cutaneous disorders obviously targets the site of disease, thereby minimizing adverse side effects elsewhere within the body. Delivery of systemic therapies via the skin avoids degradation of the medication within the gastrointestinal tract and first-pass metabolism by the liver, both of which are associated with oral administration of drugs, in addition to evading the pain and safety issues associated with injections. Transdermal delivery of drugs, in some cases, enables infrequent dosing and maintenance of steady state drug levels.

Therefore, it is desirable to provide improved clinical applications of NSAIDs through transdermal therapeutic compositions and delivery systems, in particular, for the transdermal delivery of propionic and acetic acid NSAIDs across the dermis.

### SUMMARY

The present application includes transdermal formulations for the delivery of propionic and acetic acid NSAIDs to a subject. In some embodiments, the formulation comprises at least three phases including at least one oil phase, at least one aqueous phase and at least one external phase comprising propionic and/or acetic acid NSAIDs.

In some embodiments, the present application includes a transdermal formulation comprising:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid and at least one NSAID selected from a propionic acid NSAID and an acetic acid NSAID; and optionally
(d) at least one preservative phase.

The present application includes methods for treating one or more NSAID-responsive diseases and conditions comprising administering an effective amount of one or more of the transdermal formulations of the application to a subject in need thereof. In some embodiments, the NSAID-responsive disease or condition is selected from one or more of acute pain, chronic pain, nociceptive pain, neuropathic pain, inflammation, migraine, cancer, ankylosing spondylitis, heart disease, neurodegenerative disease and auto-immune disease.

In one embodiment, application of the transdermal formulation containing at least one NSAID results in sustained release of the NSAID.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DRAWINGS

The embodiments of the application will now be described in greater detail with reference to the attached drawings in which:

FIG. 1 shows the stability of exemplary formulation 2 comprising 1.16% diclofenac over 3 months at 45° C. for pH and viscosity evolution.

FIG. 2 shows the stability of exemplary formulation 3 comprising 4% ibuprofen over 3 months at 45° C. for pH and viscosity evolution.

FIG. 3 shows the stability of exemplary formulation 4 comprising 15% ketoprofen over 3 months at 45° C. for pH and viscosity evolution.

FIG. 4 shows the cumulative pain scores at baseline and after treatment with dose 1 and dose 2 of exemplary formulation 1 compared to oral administration and a negative control.

FIG. 5 shows the cumulative function scores at baseline and after treatment with dose 1 and dose 2 of exemplary formulation 1 compared to oral administration and a negative control.

FIG. 6 shows the difference from mean baseline pain scores of treatment with dose 1 and dose 2 of exemplary formulation 1 compared to oral administration and a negative control.

FIG. 7 shows the difference from mean baseline function scores of treatment with dose 1 and dose 2 of exemplary formulation 1 compared to oral administration and a negative control.

FIG. 8 illustrates the serum concentrations of naproxen following a seven day regimen with exemplary formulation 1 and seven day washout compared with oral naproxen.

FIG. 9 illustrates the serum concentrations of diclofenac following a seven day regimen with exemplary formulation 2 and seven day washout compared with Voltaren.

FIG. 10 illustrates the synovial fluid concentrations of diclofenac following a seven day regimen with exemplary formulation 2 and seven day washout compared with Voltaren.

FIG. 11 shows the pain scores (upper panel), function scores (middle panel) and knee measurements (bottom panel) for animals treated with a base formulation compared to no treatment.

FIG. 12 shows the pain scores for animals treated with ibuprofen by oral administration (PO) (upper panel) or a transdermal base formulation (middle panel), and compared together in lower panel.

FIG. 13 shows the joint function scores for animals treated with ibuprofen by oral administration (PO) (upper panel) or a transdermal base formulation (middle panel), and compared together in lower panel.

FIG. 14 shows the knee measurements for animals treated with ibuprofen by oral administration (PO) (upper panel) or a transdermal base formulation (middle panel), and compared together in lower panel.

FIGS. 15A and 15B are bar graphs showing the amount of serum ibuprofen after administration by oral or transdermal routes.

FIGS. 16A and 16B are bar graphs showing the amount of ibuprofen in synovial fluid after administration by oral or transdermal routes.

### DETAILED DESCRIPTION

### I. Definitions

Unless otherwise indicated, the definitions and embodiments described in this and other sections are intended to be applicable to all embodiments and aspects of the present application herein described for which they are suitable as would be understood by a person skilled in the art.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this application and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

The present description refers to a number of chemical terms and abbreviations used by those skilled in the art. Nevertheless, definitions of selected terms are provided for clarity and consistency.

As used in this application, the singular forms “a”, “an” and “the” include plural references unless the content clearly dictates otherwise. For example, an embodiment including “an agent” should be understood to present certain aspects with one compound or two or more additional compounds.

In embodiments comprising an “additional” or “second” component, such as an additional or second agent, the second component as used herein is chemically different from the other components or first component. A “third” component is different from the other, first, and second components, and further enumerated or “additional” components are similarly different.

The term “agent” as used herein indicates a compound or mixture of compounds that, when added to a formulation, tend to produce a particular effect on the formulation's properties.

The term “thickening agent” as used herein refers to a compound or mixture of compounds that adjusts the thickness of the formulation.

The term “and/or” as used herein means that the listed items are present, or used, individually or in combination. In effect, this term means that “at least one of” or “one or more” of the listed items is used or present.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The term “water soluble”, for example as in “water soluble emulsion stabilizer”, refers to a substance that has a solubility in aqueous based solutions that is sufficient for the substance to exert its desired effect at concentrations that are pharmaceutically acceptable.

The term “oil soluble”, for example as in “oil soluble emulsion stabilizer”, refers to a substance that has a solubility in oil based solutions that is sufficient for the substance to exert its desired effect at concentrations that are pharmaceutically acceptable.

“Formulation” and “pharmaceutical formulation” as used herein are equivalent terms referring to a formulation for pharmaceutical use.

The term “pharmaceutically acceptable” means compatible with the treatment of animals, in particular, humans.

The term “effective amount” as used herein means an amount sufficient to achieve the desired result and accordingly will depend on the ingredient and its desired result. Nonetheless, once the desired effect is known, determining the effective amount is within the skill of a person skilled in the art.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of an active agent and optionally consists of a single administration, or alternatively comprises a series of applications. The length of the treatment period depends on a variety of factors, such as the severity of the condition, the age of the patient, the concentration of active ingredient or agent, the activity of the compositions described herein, and/or a combination thereof. It will also be appreciated that the effective dosage of the agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for duration sufficient to treat the patient.

“Topical composition” as used herein includes a composition that is suitable for topical application to the skin, nail, mucosa, wound bed or wound cavity. A topical composition may, for example, be used to confer a therapeutic or cosmetic benefit to its user. Specific topical compositions can be used for local, regional, or transdermal application of substances.

The term “topical administration” is used herein to include the delivery of a substance, such as a therapeutically active agent, to the skin or a localized region of the body.

“Transdermal” as used herein includes a process that occurs through the skin. The terms “transdermal,” “percutaneous” and “transcutaneous” can be used interchangeably. In certain embodiments, “transdermal” also includes epicutaneous. Transdermal administration is often applied where systemic delivery of an active is desired, although it may also be useful for delivering an active to tissues underlying the skin with minimal systemic absorption.

“Transdermal application” as used herein includes administration through the skin. Transdermal application can be used for systemic delivery of an active agent; however, it is also useful for delivery of an active agent to tissues underlying the skin with minimal systemic absorption. In certain embodiments, “transdermal application” can also include epicutaneous application.

The term “sustained release,” as used herein, refers to the release of an NSAID in a transdermal formulation that occurs more slowly relative to other forms such as oral dosage forms.

The term “emollient” as used herein refers to a compound or mixture of compounds that adds or replaces natural oils in the skin, for example by maintaining the integrity of the hydrolipids of the skin.

The term “polar emollient” as used herein refers to emollient compounds, which are generally oils, having heteroatoms that differ in electronegativity. This results in a dipole moment. Typical polar oils are fatty alcohols, esters and triglycerides. While they are still water insoluble and oil-loving, these oils have unique characteristics due to their polar nature. They typically combine with higher hydrophobic lipid balance (HLB) emulsifiers to make stable emulsions, they dissolve materials that are insoluble in nonpolar oils, and they provide unique properties when compared with nonpolar oils such as mineral oil.

The term “medium polar emollient” as used herein refers to emollient compounds, which are generally oils that are less polar than the polar emollients but still more polar than nonpolar oils such as mineral oil.

The term “humectant” as used herein refers to a compound or mixture of compounds intended to increase the water content of the top layers of skin.

The term “emulsifier” of “emulsifying agent” as used herein refers to a compound of mixture of compounds which promote or facilitate the dispersion of one substance in another to form an emulsion.

The term “penetration enhancer” as used herein refers to a compound or mixture of compounds that improves the rate of percutaneous transport of an active agent across the skin for use and delivery of active agents to organisms such as mammals.

The term “preservative” as used herein refers to a substance that is added to products such as pharmaceutical compositions, to prevent decomposition by microbial growth or by undesirable chemical changes. For example, the addition of antimicrobial preservatives prevents microorganism growth by modifying the pH level.

The term “flavonoid compounds” as used herein refers to a class of plant secondary metabolites that have the general structure of a 15-carbon skeleton, which contains two phenyl rings (A and B) and heterocyclic ring (C). The basic chemical structure of a flavonoid as used herein is as follows:

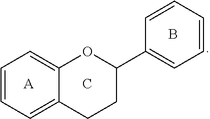

However, the term flavonoid includes the following flavonoids:

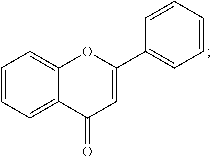 [SMILES: O=c1cc(-c2ccccc2)oc2ccccc12]

isoflavonoids:

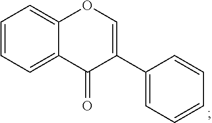 [SMILES: O=c1c(-c2ccccc2)coc2ccccc12]

and
neoflavonoids:

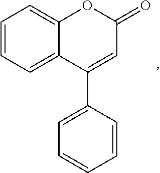 [SMILES: O=c1cc(-c2ccccc2)c2ccccc2o1]

as well as their non-ketone containing counterparts, known as flavanoids. Flavonoids are one of the largest known nutrient families, and include over 6,000 already-identified family members. Some of the best-known flavonoids include rutin, quercetin, kaempferol, catechins, and anthocyanidins. This nutrient group is most famous for its antioxidant and anti-inflammatory health benefits, as well as its contribution of vibrant color to foods.

The terms “Non-Steroidal Anti-Inflammatory Drugs” or “NSAIDs” as used herein refers to a group of non-narcotic analgesics, which are categorized chemically into six distinct classes including, the anthranilic acid derivatives, enolic acid derivatives, pyrazole derivatives, salicylates, propionic and acetic acid derivatives. All NSAIDs are weak acids having a pKa in the range of 3-5. The representative class of propionic acid NSAIDs include:

### Ibuprofen

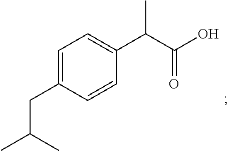

### Naproxen

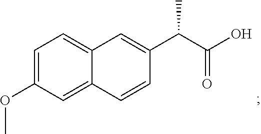 [SMILES: COc1ccc2cc([C@H](C)C(=O)O)ccc2c1]

### Fenoprofen

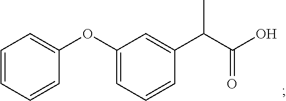 [SMILES: CC(C(=O)O)c1cccc(Oc2ccccc2)c1]

### Ketoprofen

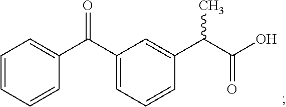 [SMILES: CC(C(=O)O)c1cccc(C(=O)c2ccccc2)c1]

### Flurbiprofen

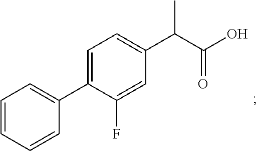 [SMILES: CC(C(=O)O)c1ccc(-c2ccccc2)c(F)c1]

### Oxaprozin

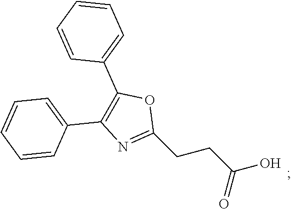 [SMILES: O=C(O)CCc1nc(-c2ccccc2)c(-c2ccccc2)o1]

### Loxoprofen

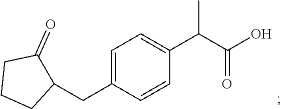 [SMILES: CC(C(=O)O)c1ccc(CC2CCCC2=O)cc1]

and includes tautomers thereof and pharmaceutically acceptable salts and solvates thereof.

The representative class of acetic acid NSAIDs include:

### Indometacin

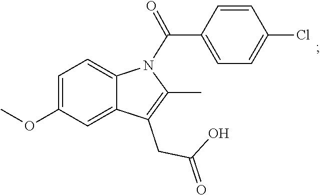 [SMILES: COc1ccc2c(c1)c(CC(=O)O)c(C)n2C(=O)c1ccc(Cl)cc1]

### Tolmetin

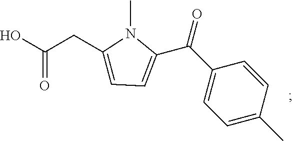 [SMILES: Cc1ccc(C(=O)c2ccc(CC(=O)O)n2C)cc1]

### Sulindac

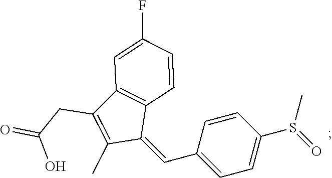 [SMILES: CC1=C(CC(=O)O)c2cc(F)ccc2/C1=C\c1ccc(S(C)=O)cc1]

### Etodolac

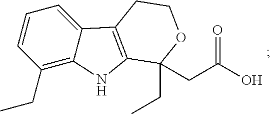 [SMILES: CCc1cccc2c3c([nH]c12)C(CC)(CC(=O)O)OCC3]

### Diclofenac

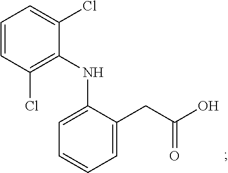 [SMILES: O=C(O)Cc1ccccc1Nc1c(Cl)cccc1Cl]

### Aceclofenac

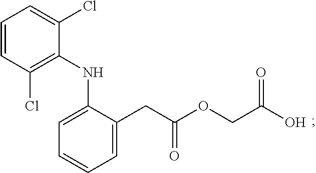 [SMILES: O=C(O)COC(=O)Cc1ccccc1Nc1c(Cl)cccc1Cl]

### Nabumetone

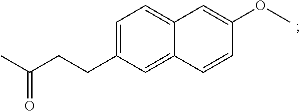 [SMILES: COc1ccc2cc(CCC(C)=O)ccc2c1]

and includes tautomers thereof and pharmaceutically acceptable salts and solvates thereof.

The term “migraine” as used herein includes migraine without aura, migraine with aura, migraine with typical aura, migraine with prolonged aura, familial hemiplegic migraine, basilar migraine, migraine aura without headache, migraine with acute onset aura, ophthalmoplegic migraine, retinal migraine, childhood periodic syndromes that may be precursors to or associated with migraine, benign paroxysmal vertigo of childhood, alternating hemiplegia of childhood, status migrainosus, and migrainous infarction.

“Acute pain” as used herein includes musculoskeletal pain, postoperative pain and surgical pain.

“Chronic pain” as used herein includes chronic inflammatory pain (e.g., rheumatoid arthritis and osteoarthritis, neuropathic pain (e.g., post herpetic neuralgia (PHN), trigeminal neuralgia, neuropathies associated with diabetes and sympathetically maintained pain) and pain associated with cancer and fibromyalgia.

The term “pharmaceutically acceptable salt” means an acid addition salt or basic addition salt which is suitable for or compatible with the treatment of subjects, including human subjects.

The term “pharmaceutically acceptable acid addition salt” as used herein means a compound formed by the reaction of a pharmaceutically acceptable acid with a basic compound. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Such salts may exist in either a hydrated, solvated or substantially anhydrous form. In general, the acid addition salts are more soluble in water and various hydrophilic organic solvents, and generally demonstrate higher melting points in comparison to their free base forms. The selection of the appropriate salt will be known to one skilled in the art. Other non-pharmaceutically acceptable acid addition salts, e.g. oxalates, may be used, for example, in the isolation of the compounds of the invention, for laboratory use, or for subsequent conversion to a pharmaceutically acceptable acid addition salt.

The term “pharmaceutically acceptable basic addition salt” as used herein means any pharmaceutically acceptable organic or inorganic base addition salt of any acid compound. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmaceutically acceptable basic addition salts, may be used, for example, in the isolation of the compounds for laboratory use, or for subsequent conversion to a pharmaceutically acceptable basic addition salt.

The term “wt %” means a percentage expressed in terms of weight of the ingredient or agent over the total weight of the formulation multiplied by 100.

The term “water” as used herein as an ingredient in the formulations of the application refers to pharmaceutically acceptable water.

### II. Formulations of the Application

In some embodiments, the transdermal formulation base of the present application comprises:
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid and at least one NSAID selected from a propionic acid NSAID and an acetic acid NSAID; and optionally
(d) at least one preservative phase.

In some embodiments, the transdermal formulation base comprises an oil-in-water emulsion. In some embodiments, the formulation is a multiphase emulsion, such as an oil-in-water-oil emulsion or a water-in-oil-water emulsion.

### Emulsifiers

In some embodiments the emulsifier is any oil-soluble fatty acid ester or mixture of fatty acid esters in which the fatty acid esters have a fatty acid composition similar to the fatty acid composition of skin for generating skin-compatible liquid crystals and to mimic the molecular organization of the intracellular lipidic laminae of the stratum corneum. Such liquid crystals are able to rapidly cross skin layers as well as to integrate into the skin's own lipid barrier to provide strength and greater integrity to this barrier.

In some embodiments the fatty acid esters are selected from sugar alcohol and fatty acid alcohol esters of any C₁₄-C₂₆-fatty acid or mixtures thereof. In some embodiments, the fatty acid esters are esters of fatty acids that are present in olive oil, palm oil and/or canola oil. In some embodiments, the fatty acids are esterified with fatty acid alcohols such as, but not limited to, cetyl alcohol, cetaryl alcohol, lauryl alcohol, stearyl alcohol, myristyl alcohol and/or oleyl alcohol. In some embodiments, the fatty acids are esterified with sugar alcohols such as, but not limited to, sorbitol, glycerol, mannitol, inositol, xylitol, erythritol, threitol, arabitol and/or ribitol. Olive oil fatty acid esters, and their use in transdermal formulations is described, for example, in U.S. Patent Application Publication No. 2011/0021439. In some embodiments, the fatty acid esters are sorbitan esters of palm oil or olive oil, such as sorbitan olivate or sorbitan palmitate. For example, sorbitan olivate is derived from fatty acids present in olive oil and esterified with sorbitol, and sorbitan palmitate is derived from fatty acids present in palm oil and esterified with sorbitol. In other embodiments, the fatty acid esters are cetearyl esters of olive oil, such as cetearyl olivate. For example, cetearyl olivate is derived from fatty acids present in olive oil and esterified with cetearyl alcohol. In further embodiments, the fatty acid esters are cetyl esters of palm oil, such as cetyl palmitate. For example, cetyl palmitate is derived from fatty acid esters present in palm oil and esterified with cetyl alcohol.

In some embodiments, the emulsifier is present in the formulations of the application in an amount of about 1 wt % to about 10 wt %, about 2 wt % to about 9 wt %, or about 3 wt % to about 5 wt %.

In some embodiments, an emulsifier is optionally included in the external phase. In some embodiments, the emulsifier in the external phase is glycerin or a glycerin/soy bean extract mixture.

In some embodiments, the emulsifier in the external phase is present in the formulations of the application in an amount of about 0.5 wt % to about 2 wt %.

### Emulsion Stabilizers

In some embodiments, the emulsion stabilizer is any compound or mixture of compounds that helps to maintain the oil-in-water emulsion. There are three types of emulsion instability: flocculation, coalescence and creaming. Flocculation describes the process by which the dispersed phase comes out of suspension in flakes. Coalescence is another form of instability, which describes when small droplets combine to form progressively larger ones. Emulsions can also undergo creaming, which is the migration of one of the substances to the top or bottom (depending on the relative densities of the two phases) of the emulsion under the influence of buoyancy or centripetal force when a centrifuge is used. Generally, emulsion stability refers to the ability of an emulsion to resist change in its properties over time. In the present application an emulsion stabilizer is present in both the oil phase and the aqueous phase.

In some embodiments, the oil soluble emulsion stabilizer is one or more waxes. In some embodiments the waxes are selected from animal and plant waxes and mixtures thereof. In some embodiments, the plant wax is a wax derived from berries, olives or from palm (e.g. carnauba wax). In some embodiment, the animal wax is beeswax. The one or more waxes are stabilizers that are present in the oil phase of the formulation.

In some embodiment, the oil soluble emulsion stabilizer is present in the formulation in an amount of about 0.5 wt % to about 5 wt % or about 1 wt % to about 4 wt %.

In some embodiments, the water soluble emulsion stabilizer is one or more thickening agents. In some embodiments, the thickening agents are any compound or mixture of compounds that maintains components in the formulation in suspension and provides a suitable consistency to the formulation.

In some embodiments, the water soluble emulsion stabilizer is selected from natural polymers, gums and synthetic polymers, and mixtures thereof. In some embodiments, natural polymers, gums and synthetic polymers, and mixtures thereof, are water soluble and therefore are present in the aqueous phase of the formulation. In some embodiments, the natural polymers are selected from alginic acid and derivatives thereof, cellulose and derivatives thereof and scleroglucans, and mixtures thereof. In some embodiments, the gums are selected from xanthan gum, tara gum, guar gum and arabic gum, and mixtures thereof. In some embodiments, the synthetic polymers are selected from polyacrylates, polyisobutenes and polysorbates, and mixtures thereof.

In some embodiments, the water soluble emulsion stabilizer is present in the formulations of the application in an amount of about 0.1 wt % to about 1 wt %, about 0.2 wt % to about 0.8 wt %, or about 0.3 wt % to about 0.7 wt %.

### Emollient Comprising at Least One Flavonoid

In some embodiments, the one or more emollients comprising one or more flavonoid compounds are polar emollients. Polar emollients generally include natural oils and extracts from plants. In some embodiments, the polar emollients are derived from fruits (including berries), vegetables, herbs, spices, legumes, leaves, seeds and/or grains. In some embodiments, the polar emollient is a natural oil or extract from citrus, Ginkgo biloba, tea, wine, cacao, onion, kale, parsley, red beans, broccoli, endive, celery, cranberries, blackberries, red raspberries, blackcurrants, acai, blueberries, bilberries, milk thistle, apples, hawthorn, Echinacea, grapes, and/or soy. In some embodiments, the polar emollient is emu oil.

In some embodiments, the polar emollient comprising one or more flavonoid compounds is a natural oil or extract from the genera Rubus, Ribes, Argania, Nymphaea, Peucedanum or Imperatoria, Sambucus, Calendula, Butea, Citrus (e.g. lime), or species or subspecies thereof. In some embodiments, the polar emollient comprising one or more flavonoid compounds comprises Leptospermum Scoparium and/or manuka oil. In some embodiments, the polar emollient comprising one or more flavonoid compounds comprises Argan oil, Sea buckthorn oil, Cicatrol, Protectol, and/or Calendula.

In some embodiments, the emollients comprising one or more flavonoid compounds are present in the formulations of the application in an amount of about 1 wt % to about 20 wt %, about 2 wt % to about 10 wt %, or about 3 wt % to about 7 wt %.

### Further Emollients

The polarity of the emollients used in the present can vary depending on the identity of the emulsifiers and emulsion stabilizers, however can nonetheless be selected by a person skilled in the art. In some embodiments, the formulations of the present application comprise both polar emollients and medium polar emollients.

In some embodiments, further polar emollients used in the present application comprise an oil from an animal in the family Dromaius, for example Dromiceius (emu) or a plant, such as, Jojoba oil, Olive oil and/or coconut oil.

In some embodiments the one or more further polar emollients are present in an amount of about 0.5 wt % to about 10 wt %, about 1 wt % to about 7 wt %, or about 2 wt % to about 6 wt %.

In some embodiments, the medium polar emollient is an ester such as octyl palmitate, isopropyl stearate and isopropyl palmitate, or an alcohol such as octyl dodecanol, or mixtures thereof.

In some embodiments the emollients also act as a thickener (stabilizer) and/or a humectant.

In some embodiments, the one or more medium polar emollients are present in an amount of 0.5 wt % to about 10 wt %, about 1 wt % to about 7 wt %, or about 1.5 wt % to about 5 wt %.

### Flavonoid-Containing Extract

In some embodiments, the one or more flavonoid-containing extracts water phase is any suitable water soluble natural extract comprising a flavonoid with anti-inflammatory and/or antioxidant properties. In some embodiments, the one or more flavonoid-containing extracts are plant-based extracts, including but not limited to, one or more of Nymphaea caerulea flower extract, Peucedanum ostruthium leaf extract, Sambuscus nigra extract, Calendula flower Extract, Gingko biloba extract, Imperatoria Alpaflor extract, Sambucus Alpaflor extract, Blue lotus extract, Calendula Alpaflor extract, Masterwort extract, Elderberry extract, Angelica extract, green tea extract, chamomile extract, pomegranate pericarp and Peucedanum ostruthium leaf extract.

In some embodiments, the one or more flavonoid-containing extracts for the external phase are present in an amount of about 0.5 wt % to about 12 wt %, about 1 wt % to about 10 wt %, or about 2 wt % to about 7 wt %.

### Phospholipid-Complexed Flavonoid

In some embodiments, the flavonoid in the phospholipid-complexed flavonoid is a bioflavonoid isolated from plants such as, but not limited to, Gingko bilboa, Crataegus sp., Passiflora incarnata, Tormentilla potentilla, Tea sinensis., Aurantium sp., Citrus sp., Eucaliptus sp., Matricaria chamomilla, Rheum sp. and Fagara sylanthoides. In some embodiments, the flavonoid is isolated from green tea, buckwheat, the leaves and petioles of asparagus, fruit of the Fava D-Ante tree, fruits and fruit rinds, for example from citrus fruits such as orange, grapefruit, lemon and lime, and berries such as mulberries and cranberries. In some embodiments, the flavonoid is selected from quercetin, myrcetin, apigenin and rutin, and mixtures thereof.

In some embodiments, the phospholipid is any phospholipid, or mixture of phospholipids, from a plant or animal, or any synthetic phospholipid. In some embodiments, the phospholipid is selected from a phosphatidylcholine, a phosphatidylethanolamine, a phosphatidylinostinol, a phosphatidylserine and lecithin, and mixtures thereof.

In some embodiments, the phospholipid-complexed flavonoid is commercially available. In some embodiments, the phospholipid-complexed flavonoid is prepared by combining the phospholipid and flavonoid in a suitable solvent or mixture of solvents, in a mole ratio of phospholipid:flavonoid of about 0.5 to 2, or about 1, and isolating the resulting complex, for example, but removal of the solvent(s), precipitation and/or lyophilization.

In some embodiments, the phospholipid-complexed flavonoid is present in an amount of about 0.5% wt % to about 5 wt %, about 1 wt % to about 4 wt %, or about 1.5 wt % to about 2.5 wt %.

Complexes of bioflavonoids with phospholipids, their preparation and use, are described, for example in U.S. Pat. No. 5,043,323, the contents of which are incorporated by reference in their entirety.

### Propionic Acid NSAIDs

In some embodiments, the propionic acid NSAID is selected from one or more of ibuprofen, naproxen, fenoprofen, ketoprofen, flurbiprofen, oxaprozin and loxoprofen. In some embodiments, the propionic acid is ibuprofen. In some embodiments, the propionic acid is naproxen. In some embodiments, the propionic acid is ketoprofen.

In some embodiments, the propionic acid NSAID is present in the formulation in an amount of about 0.5 wt % to about 25 wt %, about 1 wt % to about 20 wt %, or about 1.5 wt % to about 18 wt %.

In some embodiments, the one or more propionic acid NSAIDs are in the external phase.

### Acetic Acid NSAIDs

In some embodiments, the acetic acid NSAID is selected from one or more of indomethacin, tolmetin, sulindac, etodolac, diclofenac, aceclofenac and nabumetone. In some embodiments, the acetic acid NSAID is diclofenac. In some embodiments, the diclofenac is diclofenac sodium.

In some embodiments, the acetic acid NSAID is present in an amount of about 0.5 wt % to about 5 wt % or about 1 wt % to about 3 wt %.

In some embodiments, the one or more acetic acid NSAIDs are in the external phase.

### Water

The balance of the aqueous phase of the composition is made up of water. Further, it is an embodiment that the solvent for the external phase, the source of doxycycline and/or the preservative phase (if present) comprises water. In some embodiments, the water is purified and/or demineralized water. The purified water may, for example, be filtered or sterilized.

In some embodiments, the amount of water in the aqueous phase is about 20 wt % to about 70 wt %, or about 30 wt % to about 65 wt % (based on the total weight of the formulation).

In some embodiments, the amount of water in the external phase is about 0 wt % to about 20 wt %, or about 0 wt % to about 10 wt % (based on the total weight of the formulation).

In some embodiments, the amount of water in the preservative phase (if present) is about 0 wt % to about 5 wt % (based on the total weight of the formulation).

### Preservatives

In some embodiments, the formulations of the present application comprise at least one preservative. Preservatives include antimicrobial agents. In some embodiments the preservatives prevent or inhibit the growth of micro-organisms, including bacteria, yeasts and molds. In some embodiments, the preservatives prevent or inhibit undesirable chemical reactions from occurring. For example, in some embodiments, the preservative is an antioxidant.

In some embodiments, the preservative comprises a preservative system comprising phenoxyethanol, benzoic acid, and dehydroacetic acid. In some embodiments, the preservative comprises capryl glycol, which also advantageously has humectant and emollient properties. In some embodiments, the preservative comprises chlorphensin. In some embodiments, the preservative comprises ethylhexylglycerin which also advantageously has skin conditioning and emollient properties and acts as a deodorant. In some embodiments, the preservative comprises a natural antimicrobial agent (antibacterial, antifungal, antiviral). In some embodiments, the natural antimicrobial agent is selected from tea tree oil (Malaleuca alternifolia leaf oil) and myrtyl lemon essential oil. In some embodiments, the preservative comprises a preservative and a preservative booster.

In some embodiments, other components of the formulation have intrinsic antimicrobial properties.

In some embodiments, the one or more preservatives are present in an amount of about 0.01 wt % to about 5 wt %, about 0.5 wt % to about 4 wt %, or about 1.0 wt % to about 3 wt %.

### Further Optional Ingredients

In some embodiments, the formulations of the present application further comprise additional ingredients that are common in the transdermal base formulation art. These ingredients are, for example, but not limited to, further active pharmaceutical ingredients, pH adjusters or buffering agents, further solvents, solubilizers, chelating agents, pigments, fragrances, humectants, solubilizers, penetration enhancers, antioxidants and/or reducing agents.

(a) pH Adjusters/Buffering Agents

In some embodiments, the formulations of the application further comprise one or more pH adjusters, such as acidic, basic, or buffering components. These components may be added to provide the optimal pH balance for the skin. They may also be added to provide an optimal pH for one or more the components of the formulation. In some embodiments the pH of the formulations is adjusted to about 6 to about 7.5.

In some embodiments, the pH adjuster is selected from sodium hydroxide and potassium citrate. In some embodiment, the one or more pH adjusters are present in the formulation in an amount of about 0.05% wt % to about 2.0% wt, about 0.1 wt % to about 1.0 wt %, or about 0.8 wt % to about 0.8 wt %.

In some embodiments, the one or more pH adjusters are in the aqueous phase or the external phase.

(b) Chelating Agents

In some embodiments, the formulations of the application further comprise one or more chelating agents. In some embodiments, the chelating agents bind to metals which can inhibit the activity of the antimicrobial preservatives. In some embodiments, the chelating agent is sodium phytate or ethylendiamine tetraacetic acid (EDTA). In some embodiments, the one or more chelating agents are present in the formulation in an amount of about 0.01% wt % to about 0.2% wt, about 0.02 wt % to about 0.1 wt %, or about 0.03 wt % to about 0.05 wt %.

In some embodiments, the one or more chelating agents are in the aqueous phase or the external phase.

(c) Humectants

In some embodiments, the formulations of the present application further include one or more humectants. In some embodiments, the one or more humectants include, but are not limited to, glycerine (which also acts as an additional solvent).

In some embodiments, the one or more humectants are present in the formulation in an amount of about 0.5 wt % to about 10% wt, about 1 wt % to about 7 wt %, or about 2 wt % to about 5 wt %.

In some embodiments, the one or more humectants are in the aqueous phase.

(d) Solubilizers

In some embodiments, the formulations of the present application further include one or more solubilizers. In some embodiments, the one or more solubilizers include, but are not limited to, inulin lauryl carbamate.

In some embodiments, the one or more solubilizers are present in the formulation in an amount of about 0.01 wt % to about 5% wt.

In some embodiments, the one or more solubilizers are in the external phase.

(f) Further Active Ingredients

In some embodiments, the transdermal formulation of the present application further comprises other active ingredients. As used herein, active ingredients may include active molecules derived from natural, synthetic or semi-synthetic means, as well as other active ingredients.

In some embodiments, the further active ingredient is solubilised or dispersed in an effective amount of a suitable vehicle (e.g. solvent(s) or diluent(s)). A skilled person can readily determine which solvents or diluents will be appropriate for a particular API.

In some embodiments, the further active ingredients are selected from compounds known to treat one or more doxycycline-responsive diseases and conditions. Examples of such compound are arginine and ornithine and analogs thereof. In some embodiments, the L-arginine, L-ornithine or analogs thereof are included in the aqueous phase of the formulations of the application.

In some embodiments, the further API is included in an amount of about 0.01 wt % to about 1 wt %.

(g) Penetration Enhancer

In some embodiments the transdermal formulation of the present application further comprises penetration enhancers known in the art, for example, ethoxydiglycol (transcutanol), dimethyl isosorbide and mixtures thereof.

In some embodiments, the penetration enhancer is present in the formulation in an amount of about 1 wt % to about 30 wt % or about 3 wt % to about 20 wt %.

In some embodiments, the penetration enhancers are combined with one or more propionic acid NSAIDs in the external phase.

In some embodiments, the penetration enhancers are combined with one or more acetic acid NSAIDs in the external phase.

(h) Thickening Agents

In some embodiments, the transdermal formulation of the present application further comprises one or more thickening agents. In some embodiments, the thickening agents are present in the external phase and, in some embodiments the thickening agents enhance the stability of the formulation. In some embodiments, the thickening agents are one or more natural or synthetic polymers, such as, polyacrylates, polyisobutenes and polysorbates and mixtures thereof.

In some embodiments, the thickening agents are included in the external phase in an amount of about 0.01 wt % to about 5.0 wt % or about 0.5 wt % to about 2.0 wt %.

In some embodiments, the transdermal formulation comprises:

(a) an aqueous phase comprising water, at least one thickening agent (such as xanthan gum), and a humectant (such as glycerine);
(b) an oil phase comprising at least one emulsifier (such as cetearyl olivate, sorbitan olivate and wax stabilizers such as cetyl palmitate and sorbitan palmitate), at least one oil soluble emulsion stabilizer [wax emollient (such as carnauba wax)], at least one emollient comprising at least one flavonoid [such as natural oil or extract of red raspberries, blackcurrants, emu oil and Argan oil (polar emollient oils)], and at least one other emollient [medium polar emollient oil (such as isopropyl palmitate)];
wherein the oil and aqueous phase form an emulsion;
(c) an external phase comprising at least one flavonoid containing-extract (such as Imperatoria Alpaflor extract, Sambucus Alpaflor extract and Blue Lotus extract), at least one phospholipid-complexed flavonoid (such as lecithin and rutin), and an NSAID phase containing an NSAID and at least one penetration enhancer (such as naproxen and transcutanol);
(d) at least one preservative phase (such as phenoxyethanol, tea tree oil, chlorphenesin).

In one embodiment, the formulations of the present application are sustained release formulations. In one embodiment, the formulations are capable of achieving a stable release rate over a long period of time and stabilizes the NSAID concentration over a long period of time, for example, in the synovial fluid of a subject. In one embodiment, the formulations of the disclosure lengthen the half-life of the NSAID in the formulation.

In some embodiments, the formulations of the present application are prepared using a process that comprises:

a) heating an aqueous phase comprising water and at least one water soluble emulsion stabilizer to a first temperature;
(b) heating an oil phase comprising at least one emulsifier, at least one oil soluble emulsion stabilizer, at least one emollient comprising at least one flavonoid, and at least one other emollient to the first temperature;
(c) adding the aqueous phase to the oil phase with stirring at the first temperature and continuing to stir at the first temperature until an emulsion is formed;
(d) cooling the emulsion in (c) to a second temperature; and, in any order:
(e) adding one or more external phases comprising at least one flavonoid containing-extract, at least one phospholipid-complexed flavonoid and at least one NSAID selected from a propionic acid NSAID and an acetic acid NSAID to the emulsion at the second temperature; and optionally
(f) adding one or more preservative phases to the emulsion.

In some embodiments, the first temperature is about 65° C. to about 85° C., about 70° C. to about 80° C., or about 75° C.

In some embodiments, the second temperature is about 30° C. to about 50° C., about 35° C. to about 45° C., or about 40° C.

In some embodiments, the process further comprises preparing the external phase wherein the at least one phospholipid-complexed flavonoid is stirred with water for a sufficient amount of time to become hydrated prior to being combined with the remaining ingredients for the external phase.

In some embodiments, the phases and emulsions are mixed with a homogenizer prior to combining with other phases.

In some embodiments, the phases and emulsions are mixed with a homogenizer prior to combining with other phases.

In some embodiments of the application the formulations described herein are in the form of a cream, gel, liquid suspension, ointment, solution, patch or any other form for transdermal administration and the contents of the formulation adjusted accordingly. In some embodiments, the formulations are in the form of a cream. In some embodiments the cream has a viscosity of about 10000 cps to about 500000 cps, or about 15000 cps to about 450000 cps as measured using a Brookfield RVT T4-0.5, T4-0.6 or T4-1.5 RPM instrument at room temperature.

In some embodiments of the application, the formulation maintains its initial color, pH and/or viscosity for at least one month, at least two months or at least three months.

### III. Methods of the Application

In some embodiments, the present application includes a method for transdermal administration of one or more NSAID selected from a propionic acid NSAID and an acetic acid NSAID comprising administering an effective amount of one or more of the formulations of the present application to a subject in need thereof. In further embodiments, the present application includes a use of one or more formulations of the present application for the administration of one or more NSAID selected from a propionic acid NSAID and an acetic acid NSAID to a subject.

The present application includes therapeutic methods and uses of the formulations described herein. In some embodiments, the formulations are used in methods to treat one or more NSAID-responsive diseases and conditions.

Accordingly, the present application includes methods for treating one or more NSAID-responsive diseases and conditions, comprising administering an effective amount of a transdermal formulation of the application to a subject in need thereof. Also included is a use of a transdermal formulation of the application to treat one or more NSAID-responsive diseases and conditions. In some embodiments the NSAID-responsive disease and condition is selected from one or more of acute pain, chronic pain, nociceptive pain, neuropathic pain, inflammation, migraine, cancer, ankylosing spondylitis, heart disease, neurodegenerative disease and auto-immune disease. In some embodiments, the transdermal formulations of the application are to treat musculoskeletal pain, postoperative pain and surgical pain. In some embodiments, the transdermal formulations of the application are to treat rheumatoid arthritis, osteoarthritis, pain associated with cancer and fibromyalgia. In some embodiments, the transdermal formulations of the application are to treat breast cancer, colorectal cancer, prostate cancer and non-small cell lung cancer. In some embodiments, the transdermal formulations of the application are to treat multiple sclerosis and lupus.

In some embodiments, the formulations of the application are used in conjunction with other therapies to treat NSAID-responsive diseases and conditions.

In some embodiments, the formulations of the present disclosure are sustained release formulations, which upon application to an area, results in sustained delivery of the NSAID. In some embodiments, the transdermal formulation is applied on a once-a-day schedule, or once every other day, or once-a-week schedule.

In one embodiment, application of the transdermal formulation containing an NSAID results in sustained release of the NSAID to the target tissue after application has ceased. In a further embodiment, after application of the transdermal formulation has ceased, the NSAID is present in the target tissue, for example, synovial fluid, for at least 3 days, or at least 4 days, or at least 7 days.

### EXAMPLES

The following non-limiting examples are illustrative of the present application:

### Example 1: Preparation of Exemplary Transdermal Base Formulations Containing Propionic and Acetic Acid NSAIDs

Topical formulations comprising propionic and acetic acid NSAIDs were prepared using the ingredients listed in Tables 1, 2, 4 and 6.

### Procedure for Making Formulations 1-4

Step A: In a stainless steel container, the ingredients of Phase A were combined and heated to 75° C.

Step B: In the main tank, ingredients of Phase B were combined, ensuring the thickening agent was well dispersed. Once a homogenous solution was achieved, the solution mixture from Step A was added into the main tank, followed by rapid stirring until complete emulsification, about 2-3 minutes. The solution mixture in the main tank was gradually cooled to a reaction temperature of 35-40° C., while stirring.

Step C: In a stainless steel container, ingredients of Phase C were combined.

Step D: In a stainless steel container, ingredients of Phase D were combined.

Step E: In a stainless steel container, ingredients of Phase E were combined.

Step F: In a stainless steel container, ingredients of Phase F were combined.

Step G: While stirring, mixtures from steps C-F were added to the mixture from step B. The combined solution mixtures were stirred until homogenous and then cooled to room temperature.

### Storage Stability of Formulation 2.

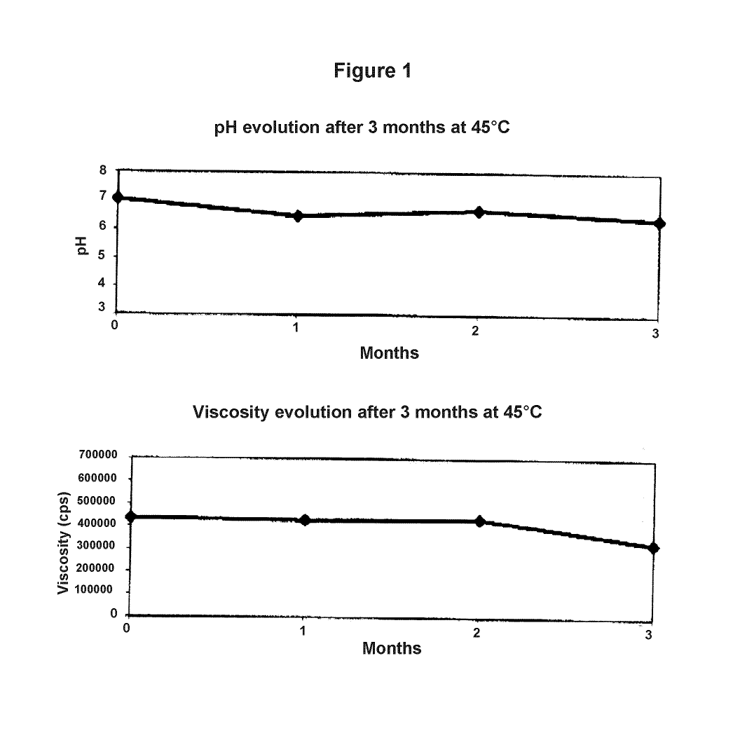

Formulation 2 was evaluated for its stability using four parameter measurements which included pH, texture, color and odour over a period of 3 months at 45° C. Formulation 2 maintained its stability in all four parameters measured providing an average pH evolution of 6.63±0.28 with a consistent viscosity evolution averaging at 406750 cps±51899 as illustrated in FIG. 1. Furthermore, the cream produced a yellow color. All measured parameters are illustrated in Table 3.

### Storage Stability of Formulation 3.

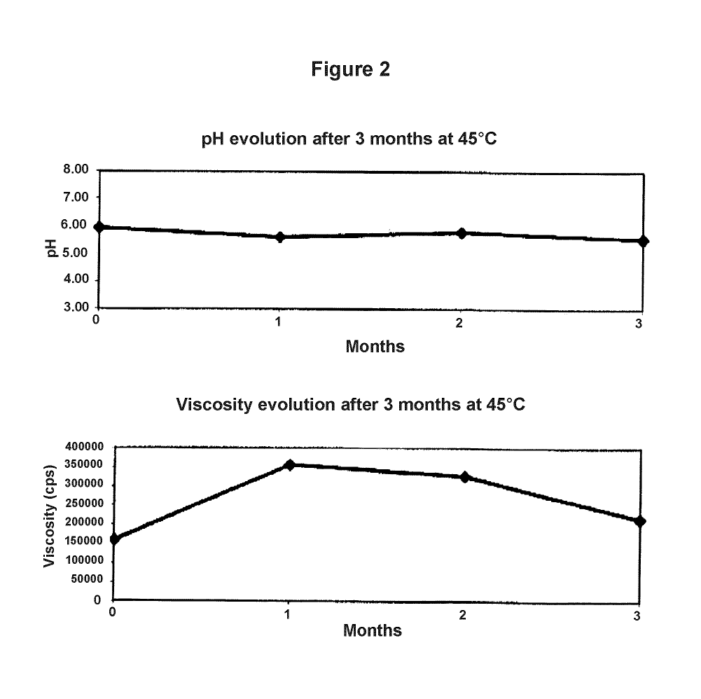

Formulation 3 was evaluated for its stability using four parameter measurements which included pH, texture, color and odour. Formulation 3 maintained its stability in all four parameters measured providing an average pH evolution of 5.72±0.17 with a consistent viscosity evolution averaging at 264700 cps±91555 as depicted in both FIG. 2 and Table 5. Furthermore, the appearance of the cream after 1 month produced oil drops on the surface and was yellow-beige in color.

### Storage Stability of Formulation 4.

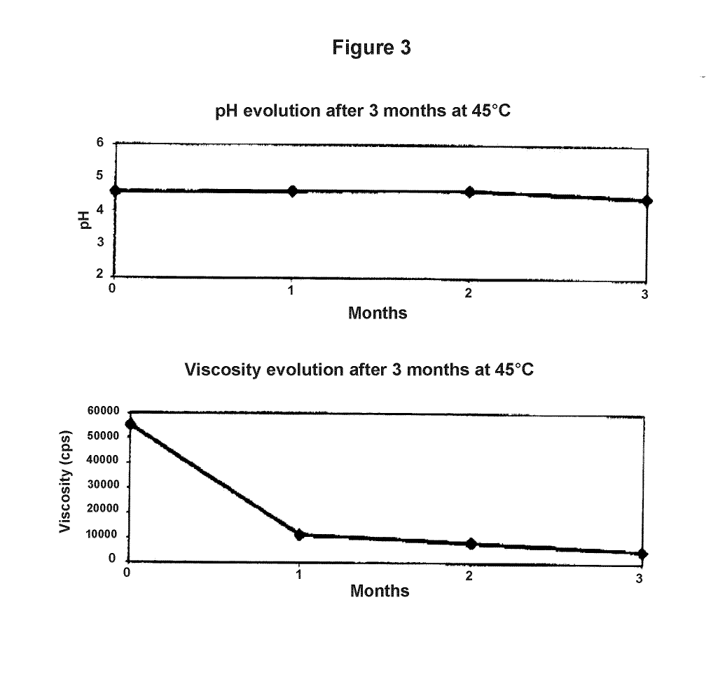

Formulation 4 was evaluated for its stability using four parameter measurements which included pH, texture, color and odour. Formulation 4 maintained its stability in all four parameters measured providing an average pH evolution of 4.56±0.09 with a decreasing viscosity evolution averaging 19975 cps±23548 as depicted in both FIG. 3 and Table 7. Furthermore, the appearance of the cream after 2 months produced oil drops on the surface and was yellow-beige in color.

### Example 2: In Vivo Evaluation of Exemplary Transdermal Formulation 1 on Measures of Pain in Aged Dogs with Osteoarthritis

Study Design

This study used a blinded parallel group design with a total of eighteen (18) aged Beagle dogs greater than six years of age with radiographic evidence of osteoarthritis. The dogs were randomized to three groups of six subjects balanced on baseline pain levels determined using a modified canine brief pain inventory questionnaire in which a technician evaluated levels of pain and functional impairment in a standardized obstacle test over two baseline days. The balanced groups then were assigned randomly to one of the following treatment conditions: 0.5 g of BID of a formulation identical to formulation 1 but without naproxen (negative control); 1 mg/kg once daily oral naproxen (positive control); and 0.5 g BID of transdermal naproxen (formulation 1, 1.5% naproxen). Dogs were tested on the modified canine brief pain inventory questionnaire daily over 7 days approximately 1.5 hours following administration of their respective treatments. Over the subsequent five test days, the same treatment doses were administered twice daily (i.e., doubling the daily dose), except that a loading dose was administered in the morning of the first day, which consisted of twice the daily dose level used initially (i.e., triple initial dose on the first day). The technicians assessing pain and function with the questionnaire were blinded to treatment conditions. Twice daily observations were conducted for the duration of the study. On the final administration day of both doses, blood was collected following the morning dose for subsequent evaluation of plasma Naproxen levels.

1) Test System

Materials and Methods

Subjects:

Eighteen (18) Beagle dogs greater than 6 years of age, with radiographic evidence of osteoarthritis were included in the study.

Housing:

Dogs were kept at an animal facility and were individually housed in metabolic cages minimally measuring 36″×36″. The animal containment room was approximately 40×80 ft with a 10-foot ceiling and consisted of a cement floor with steel walls and ceiling.

Environmental:

Environmental management including temperature, ventilation, humidity and lighting regulation in accordance with standard operating procedures. A combination of commercially acceptable fluorescent lighting and natural light was provided for the dogs. Heating and cooling was electronically controlled and was set to maintain the animal rooms in a temperature range from 15° C. to 28° C. The housing room ventilation was designed to provide a 18 filtered air changes per hour. All metabolic cages were cleaned daily.

Fees and Water:

All dogs were fed Purina Pro Plan to maintain body condition. Food was provided in stainless steel bowls at the end of each testing day. There was no difference in the feeding program between treatment groups. Water was provided via stainless steel bowls.

2) Drug Administration

Transdermal and oral naproxen were evaluated compared to a negative control as described in the Study Design. Subjects in the negative control group received ˜0.25 g of a formulation identical to formulation 1 but without naproxen, divided among the two joints with the highest radiographic scores of osteoarthritis once or twice daily. Similarly, dogs assigned to the transdermal naproxen treatment received ˜0.25 g of formulation 1 Naproxen 1.5% on their two most effected joints once or twice daily. Subjects assigned to the oral treatment received 1 mg/kg of naproxen prepared in capsules either once or twice daily. Questionnaire testing occurred approximately 1.5 hours following the morning dose administration of the test compounds. The two most affected joints of each subject were shaved prior to the first day of drug administration in all treatment groups.

3) Pain Questionnaire Assessment

Subjects were assessed on a pain questionnaire once daily on test days. The questionnaire was adapted from the canine brief pain inventory (CBPI). Specifically, the ability of each dog to walk, trot, gallop, rear, jump over a low obstacle, climb and descend stairs, jump down from a perch and general activity was evaluated in addition to a subjective evaluation of pain during evaluation of each function. Higher numerical scores reflected higher levels of pain or higher levels of functional impairment.

4) Blood Collections

On the last day each dose was administered, blood was collected immediately following the questionnaire assessment (at approximately 1.75 hours post treatment administration). The actual time of sampling was documented in the study file. Approximately 6 ml of blood was collected and placed into each K2EDTA and SST tubes. Blood tubes were centrifuged as per standard operating procedures to produce 2 aliquots of ˜1.5 ml of both plasma and serum. Samples were stored at −80° C. (±4° C.) until shipped to the sponsor on dry ice for analysis.

5) Health Observations

Twice daily observations were conducted over the course of the study as per standard operating procedures.

6) Animal Use Justification

Dogs with naturally occurring osteoarthritis are a translational model of human arthritis. Therefore, the experimental results in this canine model should confirm the likelihood of success in humans as well as the likelihood of treating canine osteoarthritis. Procedures were designed to avoid or minimize discomfort, distress and pain to the animals in accordance with the principles of the Animal for Research Act of Ontario and the guidelines of Canadian Council on Animal Care (CCAC). Because this was an exploratory study, the number of subjects (18) were chosen to ensure naproxen-related signals were detectable in a parallel design group design. Due to the variability of osteoarthritis severity in the experimental laboratory Beagle population, Beagle dogs expressing a range of osteoarthritis severity were included herein. The data derived from this study will be used to optimize the design of future studies benchmarking naproxen and determining the number of subjects required for such studies.

7) Blinding of the Study

The treatment given to each animal was not revealed to technicians collecting observational or pain questionnaire data. The experiments were blinded to all personnel in the investigation with the exception of the person(s) involved with preparing and administering the test articles, the person(s) responsible for performing subject to group allocation, and person(s) evaluating the data.

8) Calculations and Statistical Analysis

The data consisted of whole number scored for 13 questions assessing measures of both pain (0-2) and functional impairment (0-4), as well as an overall pain and function question (0-4). A cumulative pain and functional impairment score was calculated from all 12 questions for both the pain and functional impairment measures on a daily basis as applicable. Initially, repeated-measures analysis of variance (RMANOVA) were conducted for each measure with treatment group (placebo, oral naproxen and transdermal naproxen) serving as a between-subject measure and with dose (mean baseline, low dose treatment test day 5 and high dose treatment test day 5) serving as a within-subject measure. The fifth day was selected to control for potential confounding effects of measurement from multiple technicians. Subsequently, difference from the baseline scores were calculated for the fifth test day at each dose and subjected to a similar RMANOVA analysis. All statistical analyses were conducted using the Statistica 11.0 software program with significance levels set to 0.05. Post-hoc analyses were conducted using Fisher's LSD tests as appropriate. One dog in each of the naproxen groups was removed for the analysis due to the absence of measurable pain at baseline.

Animal Welfare

The Study Facility is committed to complying with all local regulations governing the care and use of laboratory animals. Procedures are designed to avoid or minimize discomfort, distress and pain to the animals in accordance with the principles of The Animal for Research Act of Ontario and the guidelines of Canadian Council on Animal Care (CCAC). The CCAC Guide for the Care and Use of Experimental Animals and related policies was regarded as guidelines to follow. To ensure compliance, this protocol was reviewed and approved by the Study Facility's Institutional Animal Care and Use Committee (IACUC) before the start of the trials as per IACUC standard operating procedures.

Animal Disposition

All animals survived the study and were returned to the Vivocore colony at study conclusion.

Results & Discussion

The objective of this study was to evaluate the effectiveness of transdermal delivery of naproxen in exemplary formulation 1 compared to oral naproxen and exemplary formulation 1 control without naproxen in treating osteoarthritic pain in a spontaneous aged dog model of osteoarthritis. Eighteen (18) Beagle dogs greater than six years of age and with radiographic evidence of osteoarthritis were included in the study.

The dogs were randomized to three groups of six subjects based on mean baseline pain levels determined using a modified canine brief pain inventory questionnaire over two baseline days. The balanced groups then were assigned randomly to the following conditions: 0.5 g once daily of formulation identical to formulation 1 but without naproxen (negative control); 1 mg/kg once daily oral naproxen (positive control); and 0.5 g once daily of transdermal Naproxen (formulation 1 with 1.5% Naproxen). Dogs were administered their respective treatments for 7 days (Dose 1), and over the subsequent five days, the same doses were administered twice daily (Dose 2). Pain was evaluated using the pain questionnaire, which was administered approximately 1.5 hours following administration of their respective treatments. The technicians assessing pain and function with the questionnaire were blinded to treatment conditions. Data from the fifth treatment day at each dose compared to baseline was statistically evaluated.

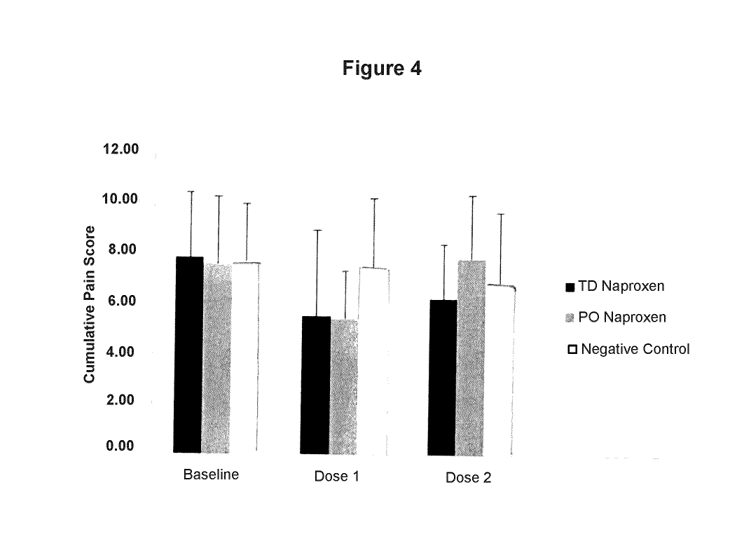

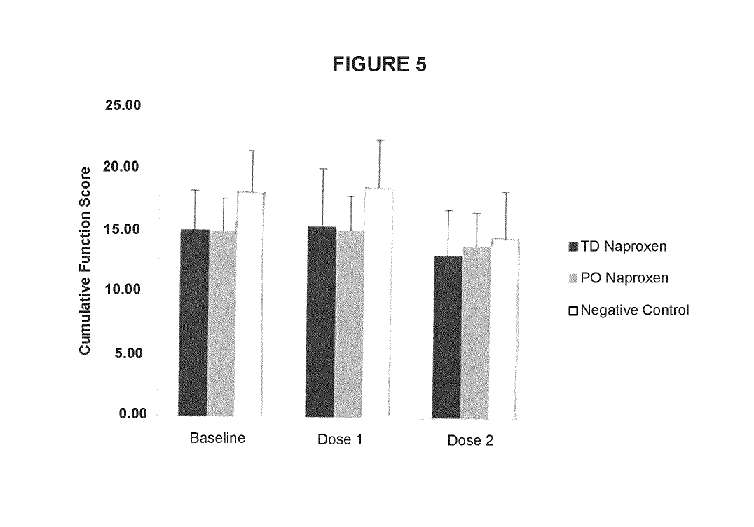

The analysis of pain scores revealed a significant [F(2,22)=11.38; p<0.001] effects of dose and a trend [F(4,22)=1.82; p0.16] interaction between treatment group and dose. The dose effect was due to significantly lower pain at the treatment time-points compared to baseline. The interaction revealed that both pain was significantly reduced from baseline with both Dose 1 and Dose 2 of the transdermal naproxen and a significant decrease from baseline was also evident following daily low dose treatment of oral naproxen (FIG. 4). The analysis of cumulative functional impairment scores revealed a significant [F(2,22)=5.03; p<0.05] effect of dose, which was due to significantly lower functional impairment during the high dose treatment compared to baseline; however, this was not treatment related (FIG. 5).

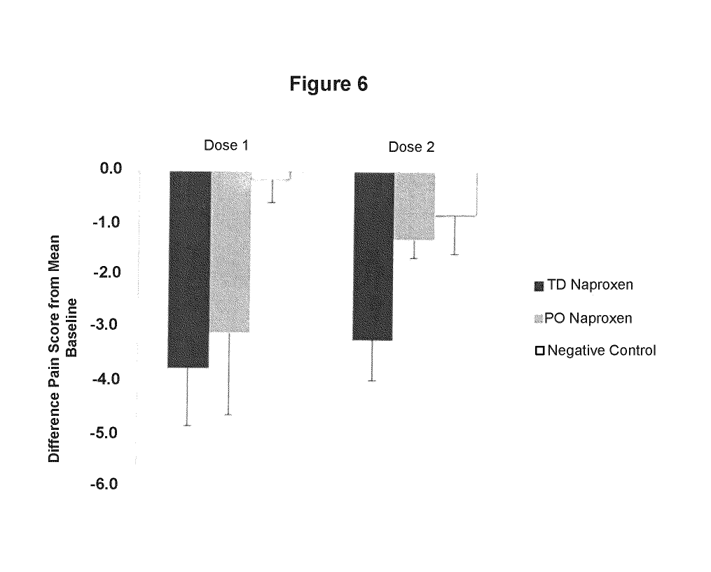

To better control for variability, differences from mean baseline scores were analyzed using a similar RMANOVA approach. The analysis of cumulative pain score difference revealed a significant [F(2,11)=14.14; p<0.001] treatment effect and a marginally significant [F(2,11)=2.95; p=0.09] interaction between treatment group and dose. Pain scores were lower under both naproxen treatments compared to the negative control, and the transdermal naproxen treatment showed a trend (p=0.12) for lower pain scores compared to the oral naproxen treatment. The interaction revealed that transdermal naproxen resulted in significantly lower pain compared to the negative control at both Dose 1 and Dose 2. By contrast, only the low dose (one dose per day) of naproxen resulted in significantly less pain compared to the negative placebo control (FIG. 6). Lastly, transdermal naproxen resulted in significantly lower pain scores at the high dose compared to oral naproxen.

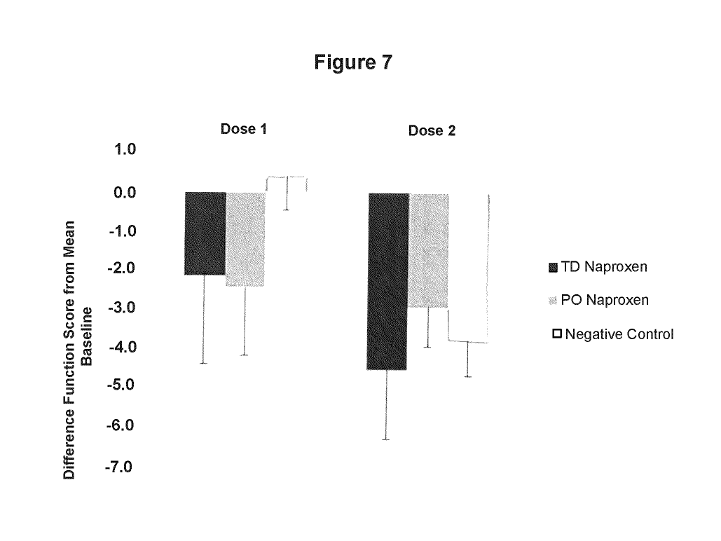

The analysis of cumulative functional impairment score difference from mean baseline revealed a significant [F(1,11)=5.39; p<0.05] effect of dose which was due to lower levels of functional impairment at the high dose (two doses per day) compared to low (one dose per day), but this was not associated with any treatment (FIG. 7).

In the current study, 2 episodes of vomition were recorded in the oral naproxen group in two dogs and three loose stool observations were recorded in the same dog receiving the transdermal naproxen, which indicates all treatments were well tolerated, and possible that the transdermal form of naproxen was better tolerated than the oral form.

Compared to baseline, transdermal naproxen significantly reduced measures of pain at both doses whereas oral naproxen reduced measures of pain at only the low dose. When difference from mean baseline scores was examined, transdermal naproxen significantly reduced pain compared to negative control at both doses whereas oral naproxen reduced pain at only the low dose. Moreover, the reduction in pain at the high dose was significantly greater in the transdermal naproxen group compared to the oral naproxen group. No treatment related effects on the functional impairment measures were found. Moreover, three episodes of vomition were reported in the oral naproxen group, and no vomition was recorded in either of the transdermal naproxen or negative control groups.

The results of the current study demonstrate that both oral and transdermal naproxen (formulation 1) reduced measures of pain in aged dogs with osteoarthritis at the low dose (one dose per day) tested here. Under the high dose, however, the transdermal naproxen showed superior pain reduction compared to oral naproxen. Moreover, the transdermal naproxen was better tolerated than oral naproxen. Studies further examining these potential differences between the two routes of administration over longer administration periods are warranted. Regardless, the current study demonstrates that effectiveness of transdermal application of naproxen in formulation 1 at the doses tested here reduces measures of pain related to osteoarthritis in dogs.

### Example 3: Oral and Transdermal Formulation 1 Bioavailability in Canine Sera

Methods

Standards:

A stock solution of naproxen was prepared in methanol/water (70:30) at a concentration of 1 mg/mL. A 1 mg/mL stock solution of d3-naproxen was prepared in dimethyl sulfoxide. These 1 mg/mL stock solutions were used to prepare 50 μg/mL solutions of naproxen and d3-naproxen by adding 50 μL of the 1 mg/mL solution to 950 μL methanol/water (70:30). A 500 μg/mL solution of naproxen was prepared by adding 500 μL of a 1 mg/mL stock solution of naproxen to 500 μL in methanol/water (70:30). A stock solution of 1 μg/mL naproxen and 200 ng/mL d3-naproxen was prepared by adding 20 μL of 50 μg/mL solution of naproxen and 4 μL of 50 μg/mL solution of d3-naproxen to 976 μL of methanol/water (70:30) (stock solution A). A solution of 200 ng/mL of d3-naproxen was prepared by adding 4 μL of 50 μg/mL solution of d3-naproxen to 996 μL methanol/water (70:30) (stock solution B). Stock solution A was serially diluted with stock solution B to give concentrations of 1000.0 ng/mL, 500.0 ng/mL, 250.0 ng/mL, 125.0 ng/mL, 62.5 ng/mL, 31.3 ng/mL, 15.6 ng/mL, 7.8 ng/mL, 3.9 ng/mL of naproxen and a constant concentration of 200 ng/ml d3-naproxen. The serial dilutions were quantified using the ratio of the peak area of naproxen to the peak area of d3-naproxen as the assay parameter. Peak area ratios were plotted against naproxen concentrations and standard curves in the form of y=A+Bx were calculated using weighted least squares linear regression.

HPLC-UV Instrumentation and Conditions:

Isocratic chromatographic separation was performed on a Zorbax Eclipse XDB-C18 column (150×4.6 mm I.D., 5 μm particle size, Agilent, Santa Clara, Calif., USA, SN USKH095544) with a XDB-C18 guard column (12.5×4.6 mm I.D., μm particle size, Agilent, Santa Clara, Calif., USA, SN USLE034730) using a mobile phase of acetonitrile/0.02 M NH₄OAc (pH=4.00) (70:30) at a flow rate of 0.500 mL/min for 6 min. The naproxen and d3-naproxen eluted between 4.37-4.45 min. There was a post time of 0.10 min. The column temperature was 40° C. and the autosampler temperature was maintained at 4° C. The sample injection volume was 10 μL. A 4000 Q trap from AB Sciex Instruments equipped with an electrospray ionization (ES1) was used in the negative ion mode with multiple reaction monitoring (MRM) for the quantitative analysis. Nitrogen was used as the collision gas and the curtain gas. The curtain gas was 10.00 psi, the collision gas was 8, the ion spray voltage was −4500 volts, the temperature was 500° C., and gas sources 1 and 2 were 40 psi. The declustering potential was −40 volts, the entrance potential was −10.00 volts, the focusing lens 1 was 10.50 volts, the collision energy was −10 volts and the cell exit potential was −20 volts. Quantification was performed using the transitions m/z 229→185 for naproxen and m/z 232→188 for d-3 naproxen with a scan time of 100 msec per transition. Analytical data were acquired and quantification processing was performed by using Analyst software.

Canine Biological Samples:

All in vivo animal husbandry, treatment regimens and sample collection were completed by InterVivo, a contract research organization. Staff involved in observation, sample collection and sample analysis were blinded to treatment conditions.

Six aged Beagle dogs (>6 years) of both sexes with quantified radiographic evidence of osteoarthritis were used for the study. Dogs (n=8) were treated with transdermal naproxen (1.5% naproxen in formulation 1, 1 g/shoulder per shoulder twice daily for 7 days prior to sample collection—˜1.5 mg naproxen per shoulder joint once daily), dogs (n=6) were give oral naproxen (6 and 30 mg naproxen in capsule PO once daily for seven days prior to collection) and dogs (n=4) were treated with the formulation 1 once daily for 7 days. On day seven serum samples were collected 60 minutes post drug administration. Whole blood samples were collected from the dogs following 7 days of treatment and 7 days post treatment. The samples were centrifuged and the serum was separated into 1 mL aliquots and stored at −80° C.

Canine Sera Samples: 2×100 μL of canine sera from the animal study was aliquoted into 1.7 mL polypropylene microtubes (MCI-175-C, Catalog no. 311-04-051, Axygen) and extracted with 200 μL of acetonitrile containing 4 μg/mL d3-naproxen. The samples were vortexed for 10 sec (VWR analog vortex mixer) and then centrifuged for 10 minutes at 3000 rpm at room temperature using Eppendorf centrifuge 5430. 40 μL, of the supernatant was removed and diluted with 760 μL of 0.02 M NH₄OAc (pH=4.00) in 1.7 mL polypropylene microtubes (MCT-175-C, Catalog no. 311-04-051, Axygen) and 120 μL, of transferred to HPLC vials (Agilent, product number 5182-0716, Agilent) with inserts (product number 5181-1270, Agilent) and capped with HPLC caps (product number 5182-0720, Agilent) and 10 μL was injected into the LC-MS/MS.

Doped Serum Control Samples:

990 μL, of human sera was spiked with 10 μL of a 500 μg/mL of naproxen. 3×100 μL of this sera was aliquoted into 1.7 mL polypropylene microtubes (MCT-175-C, Catalog no. 311-04-051, Axygen) and extracted with 200 μL of acetonitrile containing 4 μg/mL d3-naproxen. The samples were vortexed for 10 sec (VWR analog vortex mixer) and then centrifuged for 10 minutes at 3000 rpm at room temperature using Eppendorf centrifuge 5430. 40 μL of the supernatant was removed and diluted with 760 μL of 0.02 M NH₄OAc (pH=4.00) in 1.7 mL polypropylene microtubes (MCT-175-C, Catalog no. 311-04-051, Axygen) and 120 μL of transferred to HPLC vials (Agilent, product number 5182-0716, Agilent) with inserts (product number 5181-1270, Agilent) and capped with HPLC caps (product number 5182-0720, Agilent) and 10 μL was injected into the LC-MS/MS.

Spiked Serum Control Samples:

3×100 μL of human sera was aliquoted into 1.7 mL polypropylene microtubes (MCT-175-C, Catalog no. 311-04-051, Axygen) and extracted with 200 μL of acetonitrile containing 4 μg/mL of d-3 naproxen and 4 μg/mL of naproxen (prepared by adding 80 μL of 50 μg/mL d3-naproxen and 80 μL of 50 ug/mL naproxen to 840 μL of acetonitrile). The samples were vortexed for 10 sec (VWR analog vortex mixer) and then centrifuged for 10 minutes at 3000 rpm at room temperature using Eppendorf centrifuge 5430. 40 μL, of the supernatant was removed and diluted with 760 of 0.02 M NH₄OAc (pH=4.00) in 1.7 mL polypropylene microtubes (MCT-175-C, Catalog no. 311-04-051, Axygen) and 120 μl, of transferred to HPLC vials (Agilent, product number 5182-0716, Agilent) with inserts (product number 5181-1270, Agilent) and capped with HPLC caps (product number 5182-0720, Agilent) and 10 μL was injected into the LC-MS/MS.

Blank Serum Control Samples:

3×100 μL, of human sera was aliquoted into 1.7 mL polypropylene microtubes (MCT-175-C, Catalog no. 311-04-051, Axygen) and extracted with 200 μL of acetonitrile containing 400 μg/mL of d-3 naproxen. The samples were vortexed for 10 sec (VWR analog vortex mixer) and then centrifuged for 10 minutes at 3000 rpm at room temperature using Eppendorf centrifuge 5430. 40 μL of the supernatant was removed and diluted with 40 μL of 0.02 M NH₄OAc (pH=4.00) in 1.7 mL polypropylene microtubes (MCT-175-C, Catalog no. 311-04-051, Axygen) and 120 μL of transferred to HPLC vials (Agilent, product number 5182-0716, Agilent) with inserts (product number 5181-1270, Agilent) and capped with HPLC caps (product number 5182-0720, Agilent) and 10 μL was injected into the LC-MS/MS.

Results & Discussion

To determine if formulation 1 is capable of introducing naproxen through the subcutaneous tissue and enter the systemic circulation and synovial fluid, a canine comparative experiment was conducted, using transdermal and oral naproxen.

Animals treated with oral naproxen had an average concentration of 102.4+/−2.1 μg/mL (mean+/−SEM) after the seven day regimen and an average concentration of 16.2+/−0.8 μg/mL (mean+/−SEM) seven days post-treatment. Animals treated with naproxen in formulation 1 has an average concentration of 45.6+/−4.2 ng/mL (mean+/−SEM) after the seven day regimen and an average concentration of 9.8+/−0.9 ng/mL (mean+/−SEM) seven days post-treatment.

Control concentrations of naproxen were within acceptable ranges with spiked naproxen concentration of 92.5% % for sera indicating a small ionization depressing matrix effect between standards and unknown samples (data not shown). Control concentrations of doped naproxen demonstrated an extraction efficiency of 98% from sera indicating an acceptable extraction procedure (data not shown).

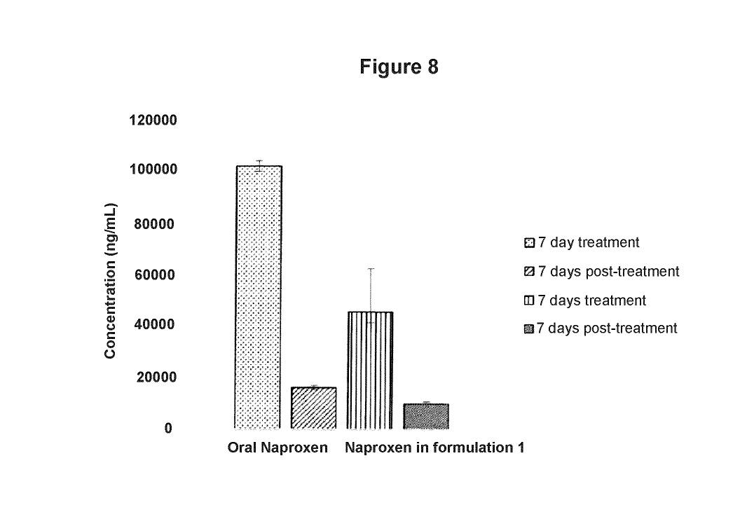

The naproxen quantification results are illustrated for serum (FIG. 8) for both oral and transdermal (formulation 1) routes of administration. With respect to naproxen concentrations in dog sera, naproxen was detected (FIG. 8) in all samples in animals that were treated with oral and transdermal naproxen after the seven day administration regimen. In addition, naproxen was detected in the sera (FIG. 8) of animals treated with oral and transdermal naproxen following a seven day post-treatment (washout) period. Additionally, high levels of naproxen were detected in the sera of animals treated with the formulation 1 base. This is likely due to the previous treatments of oral and transdermal naproxen that had not cleared from circulation.

The data supports the hypothesis that a transdermal formulation 1 product is capable of introducing naproxen into the circulatory system of a pre-clinical animal. The levels of naproxen measured in serum samples for animals treated with oral naproxen was higher than those serum samples of animals treated with transdermal naproxen.

### Example 4: Quantification of Formulation 2 in Canine Sera Samples

Methods

Standards:

A stock solution of diclofenac was prepared in acetonitrile/methanol (90:10) at a concentration of 1 mg/mL. A 1 mg/mL stock solution of d4-diclofenac was prepared in dimethyl sulfoxide. These 1 mg/mL stock solutions were used to prepare 50 μg/mL solutions of diclofenac and d4-diclofenac by adding 50 μL of the 1 mg/mL solution to 950 μL acetonitrile/methanol (90:10). A 100 μg/mL solution of diclofenac was prepared by adding 200 μL of a 1 mg/mL stock solution of diclofenac to 800 μL in acetonitrile/methanol (90:10). A 1 μg/mL of diclofenac was prepared by adding 20 μL of a 1 mg/mL stock solution of diclofenac to a 980 μL in acetonitrile/methanol (90:10). A stock solution of 1 μg/mL diclofenac and 125 ng/mL of d4-diclofenac was prepared by adding 20 μL of 50 μg/mL solution of diclofenac and 2.5 μL of 50 μg/mL of d4-diclofenac to 977.5 μL of acetonitrile/water/acetic acid (50:50:0.1) (stock solution A). A solution of 125 ng/mL of d4-diclofenac was prepared by adding 2.5 μL of 50 μg/mL solution of d4-diclofenac to 997.5 μL acetonitrile/water/acetic acid (50:50:0.1) (stock solution B). Stock solution A was serially diluted with stock solution B to give concentrations of 1000.0 ng/mL, 500.0 ng/mL, 250.0 ng/mL, 125.0 ng/mL, 62.5 ng/mL, 31.3 ng/mL, 15.6 ng/mL, 7.8 ng/mL, 3.9 ng/mL of diclofenac and a constant concentration of 125 ng/ml d4-diclofenac. The serial dilutions were quantified using the ratio of the peak area of diclofenac to the peak area of d4-diclofenac as the assay parameter. Peak area ratios were plotted against naproxen concentrations and standard curves in the form of y=A+Bx were calculated using weighted least squares linear regression.

Voltaren™ (Emugel™) is a whitish, soft, homogenous, cream-like oil-in-water topical emulsion contains diclofenac diethylamine 1.16% w/w. Nonmedicinal ingredients are carbomer, cocoyl caprylocaprate, diethylamine, isopropyl alcohol, liquid paraffin, macrogol cetostearyl ether, perfume, propylene glycol, and purified water.

HPLC-UV Instrumentation and Conditions:

Isocratic chromatographic separation was performed on a Zorbax Eclipse XDB-C18 column (150×4.6 mm I.D., 5 μm particle size, Agilent, Santa Clara, Calif., USA, SN USKH095544) with a XDB-C18 guard column (12.5×4.6 mm I.D., μm particle size, Agilent, Santa Clara, Calif., USA, SN USLE034730) using a mobile phase of acetonitrile/water/acetic acid (80:20:0.1) at a flow rate of 0.500 mL/min for 6 min. The diclofenac and d4-diclofenac eluted between 5.20-5.60 min. There was a post time of 0.10 min. The column temperature was 40° C. and the autosampler temperature was maintained at 4° C. The sample injection volume was 10 μL. A 4000 Q trap from AB Sciex Instruments equipped with an ESI was used in the positive ion mode with MRM for the quantitative analysis. Nitrogen was used as the collision gas and the curtain gas. The curtain gas was 10.00 psi, the collision gas was 8, the ion spray voltage was 4500 volts, the temperature was 350° C., and gas sources 1 and 2 were 40 psi. The declustering potential was 40 volts, the entrance potential was 10.00 volts, the focusing lens 1 was −10.50 volts, the collision energy was 30 volts and the cell exit potential was 20 volts. Quantification was performed using the transitions m/z 295.50 4215.8 for diclofenac and m/z 300.170→219.800 for d4-diclofenac with a scan time of 100 msec per transition. Analytical data were acquired and quantification processing was performed by using Analyst software.

Canine Biological Samples:

All in vivo animal husbandry, treatment regimens and sample collection were completed by InterVivo, a contract research organization. Staff involved in observation, sample collection and sample analysis were blinded to treatment conditions.

Six aged beagle dogs (>6 years) of both sexes with quantified radiographic evidence of osteoarthritis were used for the study. Dogs (n=6) were treated with transdermal diclofenac (1.16% diclofenac in formulation 2, 1 g/shoulder per shoulder twice daily for 7 days prior to sample collection—11.6 mg diclofenac per shoulder joint twice daily), dogs (n=6) were treated with transdermal diclofenac (1.16% diclofenac in Emugel™ (Voltaren™), 1 g/shoulder per shoulder twice daily for 7 days prior to sample collection—˜11.6 mg diclofenac per shoulder joint twice daily), and dogs (n=6) were treated with a formulation identical to formulation 2 but without diclofenac twice daily for seven days.

Whole blood samples were collected from the dogs following seven days of treatment and seven days post treatment. Collections for all experiments will occur 1.0 hour following treatment (±15 minutes). Collections will occur at the same time of day for each subject (±90 minutes). Subsequently, animals were maintained untreated for seven days after which serum samples were collected.

Blood will be collected from a suitable vein as per standard operating procedures into K2EDTA tubes and centrifuged as per standard operating procedures. Serum and plasma will be separated into aliquots of approximately 1 ml, which will be stored at approximately −80° C. until shipped to the sponsor on dry ice for analysis.

Baseline synovial fluid will be collected from subjects on study Days −1, 13, 27, 42, 55, 69, 83, 97 and 111, and on the final day of each treatment week (Days 6, 20, 34, 48, 62, 76, 90, 104, & 118). Synovial fluid will be collected under sedation. Subjects will be sedated with medetomidine (0.01-0.03 mg/kg) and butorphenol (0.1-0.3 mg/kg) IV. Sedation will be reversed with atipamazole (0.1-0.2 mg/kg IM). The fluid will be collected by arthrocentesis into the joint following surgical preparation of the area. Baseline and treatment collections will occur from both shoulders with the goal to collect a minimum of 0.4 mL of fluid combined across the 2 joints. Baseline collections will be repeated the subsequent day prior to treatment administration if the minimum volume is not obtained. If collection volumes are not adequate on treatment days, repeat collections will occur on the first day assigned to washout following treatment administration if applicable.

Doped Serum Control Samples:

990 μL, of human sera was spiked with 10 μL of a 50 μg/mL of diclofenac. 3×150 μL, of this sera was aliquoted into 1.7 mL polypropylene microtubes (MCT-175-C, Catalog no. 311-04-051, Axygen) and extracted with 375 μL of acetonitrile. The samples were vortexed for one minute and then centrifuged for ten minutes at 11, 000 rpm. 395 μL of supernatant was removed and placed in an insert (product number 5181-1270, Agilent) in HPLC vials (Agilent, product number 5182-0716, Agilent) and capped with HPLC caps (product number 5182-0720, Agilent). 5 μL, of 10 μg/mL of d4 diclofenac was added.

Spiked Serum Control Samples:

3×150 μL of human sera was aliquoted into 1.7 mL polypropylene microtubes (MCT-175-C, Catalog no. 311-04-051, Axygen) and extracted with 375 μL of acetonitrile. The samples were vortexed for one minute and then centrifuged for ten minutes at 11, 000 rpm. 390 μL of supernatant was removed and placed in an insert (product number 5181-1270, Agilent) in HPLC vials (Agilent, product number 5182-0716, Agilent) and capped with HPLC caps (product number 5182-0720, Agilent). 5 μL of 10 μg/mL of d4 diclofenac and 5 μL of 10 μg/mL of diclofenac was added.

Blank Serum Control Samples:

3×150 μL of human sera was aliquoted into 1.7 mL polypropylene microtubes (MCT-175-C, Catalog no. 311-04-051, Axygen) and extracted with 375 μL of acetonitrile. The samples were vortexed for one minute and then centrifuged for ten minutes at 11, 000 rpm. 390 μL of supernatant was removed and placed in an insert (product number 5181-1270, Agilent) in HPLC vials (Agilent, product number 5182-0716, Agilent) and capped with HPLC caps (product number 5182-0720, Agilent).

Unknown Serum Samples:

3×150 μL of this sera was aliquoted into 1.7 mL polypropylene microtubes (MCT-175-C, Catalog no. 311-04-051, Axygen) and extracted with 375 μL of acetonitrile. The samples were vortexed for one minute and then centrifuged for ten minutes at 11, 000 rpm. 395 μL of supernatant was removed and placed in an inserts (product number 5181-1270, Agilent) in HPLC vials (Agilent, product number 5182-0716, Agilent) and capped with HPLC caps (product number 5182-0720, Agilent). 5 μL of 10 μg/mL of d4 diclofenac was added.

Doped Synovial Fluid Control Samples:

90 μL of canine synovial fluid was spiked with 10 μL of a 10 μg/mL of diclofenac. 3×50 μL, of this synovial was aliquoted into 1.7 mL polypropylene microtubes (MCT-175-C, Catalog no. 311-04-051, Axygen), diluted with water (50 μL), and extracted with 250 μL of acetonitrile. The samples were vortexed for one minute and then centrifuged for ten minutes at 11, 000 rpm. 175 μL of supernatant was removed and placed in an inserts (product number 5181-1270, Agilent) in HPLC vials (Agilent, product number 5182-0716, Agilent) and capped with HPLC caps (product number 5182-0720, Agilent). 25 μL of 1 μg/mL of d4 diclofenac was added.

Spiked Serum Control Samples:

3×50 μL of canine synovial fluid was aliquoted into 1.7 mL polypropylene microtubes (MCT-175-C, Catalog no. 311-04-051, Axygen), diluted with water (50 μL), and extracted with 250 μL of acetonitrile. The samples were vortexed for one minute and then centrifuged for ten minutes at 11, 000 rpm. 150 μL of supernatant was removed and placed in an inserts (product number 5181-1270, Agilent) in HPLC vials (Agilent, product number 5182-0716, Agilent) and capped with HPLC caps (product number 5182-0720, Agilent). 25 μL of 1 μg/mL of d4 diclofenac and 25 μL of 1 μg/mL of diclofenac was added.

Unknown Synovial Fluid Samples:

2×50 μL (when possible) of canine synovial fluid was aliquoted into 1.7 mL polypropylene microtubes (MCT-175-C, Catalog no. 311-04-051, Axygen), diluted with water (50 μL) and extracted with 250 μL of acetonitrile. The samples were vortexed for one minute and then centrifuged for ten minutes at 11, 000 rpm. 175 μL of supernatant was removed and placed in an inserts (product number 5181-1270, Agilent) in HPLC vials (Agilent, product number 5182-0716, Agilent) and capped with HPLC caps (product number 5182-0720, Agilent). 25 μL of 1 μg/mL of d4 diclofenac was added.

Results & Discussion

The purpose of this study was to determine if formulation 2 is capable of delivering diclofenac across the epidermis of canines compared with Voltaren. The concentration of diclofenac in sera, plasma and synovial fluid was determined following treatment with diclofenac in formulation 2, treatment with a formulation identical to formulation 2 but without diclofenac (negative control), and Voltaren (positive control).

Concentration of diclofenac in sera were measured wherein the animals treated with Voltaren had an average concentration of 52.1+/−11.1 ng/mL (mean+/−SEM) after the seven day regimen and no detectable concentration seven days post-treatment. Animals treated with the diclofenac in formulation 2 had an average concentration of 78.8+/−20.0 μg/mL (mean+/−SEM) after the seven day regimen and no detectable concentration seven days post-treatment.

Concentration of diclofenac in synovial fluid was measured wherein animals treated with Voltaren had an average concentration of 438.6+/−103.2 ng/mL (mean+/−SEM) in the shoulders and 211.7+/−21.2 ng/mL (mean+/−SEM) in the stifles after the seven day regimen and no detectable concentration seven days post-treatment. Animals treated with the diclofenac in formulation 2 had an average concentration of 156.6+/−47.1 μg/mL (mean+/−SEM) in the shoulders and an average concentration of 402.3+/−4.2 μg/mL (mean+/−SEM) in the stifles after the seven day regimen. Diclofenac was detected in the shoulder and stifle of one animal seven days post-treatment (61 ng/mL and 67 ng/mL, respectively).

Control concentrations of diclofenac in sera were within acceptable ranges with spiked diclofenac concentration between 94-110% for sera (data not shown). Control concentrations of diclofenac in sera of doped diclofenac samples demonstrated an extraction efficiency between 82-99% from sera indicating an acceptable extraction procedure (data not shown).

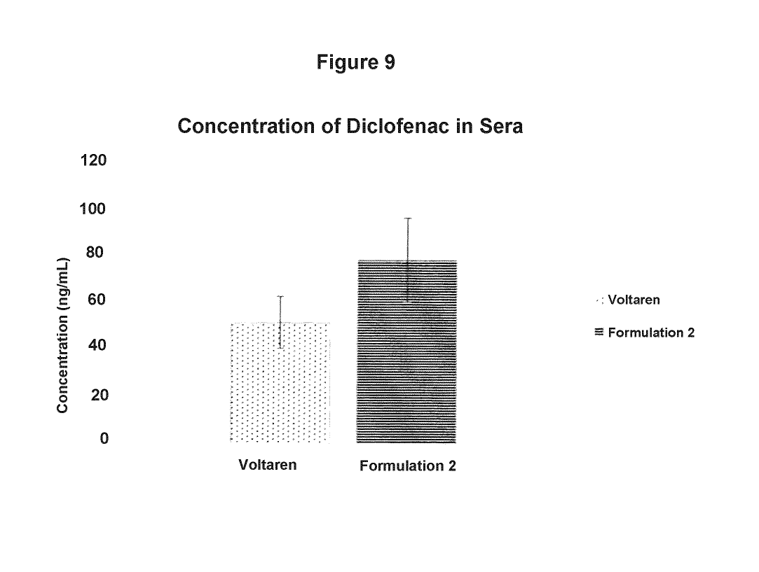

The diclofenac quantification results are illustrated for sera (FIG. 9) for both transdermal formulation 2 and transdermal Voltaren routes of administration. With respect to diclofenac concentrations in canine sera, diclofenac was detected (FIG. 9) in all samples in animals that were treated with both types of transdermal administration after the seven day administration regimen. In addition, diclofenac was not detected in the sera of animals treated with both transdermal delivery systems following a seven day post-treatment (washout) period. No diclofenac was detected in the sera of animals treated with the negative control.

Control concentrations of diclofenac were within acceptable ranges with spiked diclofenac concentration between 94-102% for synovial fluid (data not shown). Control concentrations of doped diclofenac demonstrated an extraction efficiency between 93-100% from synovial fluid indicating an acceptable extraction procedure (data not shown).

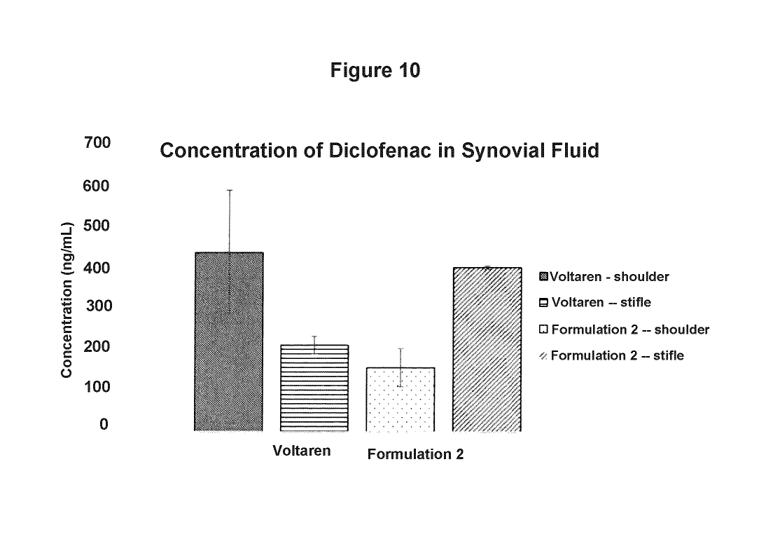

The diclofenac quantification results are illustrated for synovial fluid (FIG. 10) for both transdermal formulation 2 and transdermal Voltaren routes of administration. With respect to diclofenac concentrations in canine synovial fluid, diclofenac was detected (FIG. 10) in all samples in animals that were treated with both types of transdermal administration after the seven day administration regimen. No diclofenac was detected in the synovial fluid of animals treated with the negative control.

The data supports the hypothesis that a transdermal formulation 2 product is capable of introducing diclofenac into the synovial fluid and the circulatory system of a pre-clinical animal. The levels of diclofenac measured in serum samples for animals treated with the diclofenac in formulation 2 was slightly higher than those serum samples of animals treated with Voltaren but it was not statistically significant. The levels of diclofenac measured in the synovial fluid for animals treated with the diclofenac in formulation 2 are comparable to the levels of diclofenac in the synovial fluid for animals treated with Voltaren.

### Example 5: Efficacy of Compounded Formulation 6

A compounded transdermal formulation was prepared using the ingredients listed in Tables 8 and 9.

### Procedure for Making Formulation 5

Step A: Demineralized water and chelating agent were added to a master kettle and heated to 75° C. The solution mixture was stirred until homogenous to ensure that the chelating agent was completely hydrated.

Step B: In a separate vessel, humectant and thickener were mixed until homogenous and added to the master kettle in Step A.

Step C: In another kettle, ingredients of phase A were combined and heated to 75° C. Once a homogenous solution was achieved, the solution mixture was added into the master kettle, followed by rapid stirring until complete emulsification, about 2-3 minutes.

Step D: The solution mixture in the master kettle was gradually cooled, while stirring. When the reaction temperature reached 35-40° C., ingredients of phase C were added one by one, whereby with each addition the solution was mixed until homogenous.

Step E: In a separate vessel, the ingredients of Phase D were added and mixed. Once a homogenous solution was achieved, the solution mixture was added into the master kettle, followed by stirring until the solution became homogenous.

Step F: In a separate vessel, the ingredients of Phase F were combined until homogeneous. The resulting solution was added into the master kettle.

Step G: In a separate vessel, phospholipid complexed rutin was mixed with demineralized water. The homogenous solution was then added to the master kettle and further cooled to room temperature.

Step I: The viscosity using a Brookfield RVT, T4, 2 RPM instrument and pH measurements of the final solution were taken. The viscosity and pH values were within the range of 85,000-200,000 cps and 6.0-7.5 at 25° C., respectively.

### Procedure for Making Formulation 6

Step A: The ingredients of phase A were combined with formulation 5 followed by stirring until the solution became homogenous.

### Stability of Formulation 6

Compounded transdermal formulations of NSAIDs were explored as means to avoid some of the challenges associated with oral medications, in particular, through the reduction in systemic absorption of the drug. Generally, these formulations are prepared using either a single or combination of multiple actives in a pre-designed compounding base. In the case of the present invention, formulation 5 is the pre-designed compounding base to which the propionic acid NSAID and/or acetic acid NSAID actives are added.

Ketoprofen was compounded with the pre-designed compounding base formulation 5 to provide formulation 6. However, formulation 6 produced variability, with non-consistent results in efficacy studies, indicating compounded formulation 6 to be non-homogenous. Without wishing to be bound by theory, it is hypothesized homogeneity of the transdermal formulations of the present invention are achieved by adding the NSAID actives within the external phase of the formulation preparation.

### Example 6: Clinical Efficacy Studies of Formulation 1 and Formulation 4

13 patients participated in the clinical efficacy studies having either acute or chronic pain and associated inflammation throughout various extremities of the body including hands, shoulder, knees, neck, back, hip, ankle and face. Patients ranged in age from 40 to 90.

4 patients were treated with approximately 0.5-1.0 g of formulation 1 comprising 1.5% naproxen and 9 patients were treated with formulation 4 comprising 15% ketoprofen, wherein both exemplary formulations 1 and 4 were applied to the area of need and the outcome including a decrease in inflammation was measured. Results of the study are illustrated in Table 10.

Results & Discussion

The primary objective of this study was to assess the clinical efficacy of propionic or acetic acid NSAIDs in exemplary formulations 1 and 4 of the present application, on patients harboring acute pain, chronic pain or inflammation on their extremities.

13 patients participated in the clinical efficacy studies having either acute or chronic pain and associated inflammation throughout various extremities of the body including hands, shoulder, knees, neck, back, hip, ankle and face. Patients ranged in age from 40 to 90.

4 patients were treated with approximately 0.5 g-1.0 g of formulation 1 comprising 1.5% naproxen and 9 patients were treated with approximately 0.5 g-1.0 g of formulation 4 comprising 15% ketoprofen. Both exemplary formulations were applied to the area of need. The majority of patients treated with 1.5% naproxen had a positive response to the treatment with only one patient experiencing a short lived mild relief (Table 10). Similarly, patients treated with 15% ketoprofen had a positive response to the treatment with 3 patients unable to see a decrease in their swelling (Table 10).

Transdermal formulations comprising NSAIDs may reduce some of the common adverse effects associated with oral NSAID medications, potentially through the reduction of drug concentration, increasing absorption, maintaining the drug at target site leading to less toxicity, less clearance and greater analgesic effect. Adverse effects in clinical trials comparing transdermal and oral NSAIDs revealed that the risk of GI complications is minimal with exposure to transdermal NSAIDs as compared to oral NSAIDs. The aforementioned studies of the present application indicate the transdermal formulations of the present application reduce the systemic absorption of propionic and acetic acid NSAIDs and prove more effective at localizing and treating NSAID-responsive diseases and conditions.

### Example 7—Comparison of Oral and Transdermal Ibuprofen in Canine Model Urate Induced Acute Joint Inflammation

The objective of this study was to assess the effectiveness of oral and transdermal anti-inflammatories on canine pain responses and joint swelling in the sodium urate induced synovitis model of inflammation.

Methods

The study was a randomized, blinded, preclinical study using a controlled, parallel, matched-group design. There were 5 treatment groups, each containing 8 animals. Thirty-two subjects meeting the inclusion criteria were enrolled in the trial and allocated to 4 balanced groups; therefore, one group received 2 treatments separated by a minimum of 14 days to allow for drug washout. Following induction of synovitis by sodium urate injection, effectiveness of compounds was evaluated using a pain questionnaire assessment and joint measurements at various time points.

The following formulations were used in this study:

Group 1

| | |
|---|---|
| Name | Ibuprofen 4% in base formulation (4) |
| Dosage Form | Transdermal cream |
| Doses Tested | 4 mg/kg |
| Manufacturer | Delivra Inc. |
| Drug storage during study | Refrigerated 2-4° C. |

Group 2

| | |
|---|---|
| Name | Oral Ibuprofen |
| Dosage Form | Powder in gelatin capsule |
| Doses Tested | 4 mg/kg |
| Manufacturer | Medisca |
| Drug storage during study | Refrigerated 2-4° C. |

Group 3

| | |
|---|---|
| Name | Base formulation |
| Dosage Form | Transdermal cream |
| Doses Tested | |
| Manufacturer | Delivra Inc. |
| Drug storage during study | Refrigerated 2-4° C. |

Group 4

No Treatment

Test System

Thirty-two (32) beagle dogs obtained from the Vivocore Inc. colony were included in the study. Ages of the dogs ranged from 1.0 to 11.6 years at study initiation. There were 17 males and 15 females included.

Selection and Allocation of Animals

Animals in good general health as determined by historical health records were included in the study. Included subjects had no clinical signs of osteoarthritis and a baseline cumulative pain score of 5 or less determined using a validated pain questionnaire.

Subjects were divided into two groups based on sex. The dogs were then divided into four groups such that groups were balanced for age to the extent as possible. The four groups were then randomly assigned to treatment conditions.

Acclimation and Pre-Treatment of Test System

All animals involved in this investigation were housed at the Vivocore facility for no less than six months. Therefore, no acclimation period was needed.

Administration of Test Articles

Subjects who were scheduled for synovitis induction were treated according to their assigned treatment condition. Test article administration occurred 30 minutes following synovitis induction, i.e. sodium urate injection. Transdermal treatment doses were measured in milligrams using an analytical scale and were then applied directly to the induced stifle. Transdermal treatments were rubbed into the dog's skin for a minimum of 1 minute. Oral treatments were administered with attention to complete delivery and retention of the entire intended dose. Compounds were tested as outlined in the Schedule of Operations.

Housing and Management of Test System

Housing

Dogs were individually housed in metabolic cages in compliance with the recommendations of the Canadian Council on Animal Care. All cages and housing areas were cleaned daily. Dogs were exercised according to standard operating procedure on non-testing days and following completion of pain and inflammation assessments on test days.

Environmental

Environmental management including lighting, ventilation, temperature, and humidity regulation were maintained and controlled according to standard operating procedures as described below. A combination of commercially acceptable fluorescent lighting and natural light was provided for the dogs. Heating and cooling was electronically controlled and was set to maintain the animal room in a temperature range from 15° C. to 28° C. The housing room ventilation was designed to provide 18 filtered air changes per hour.

Feed and Water

All animals were fed according to standard operating procedure at the end of each day. Dogs were fed a standard commercial dry diet to maintain body condition. Food consumption was not recorded. Water was provided ad libitum.

Procedures and Data Recorded

Twice Daily Observations

Animals were observed twice daily over the course of the study according to facility standard operating procedures. Observational data can be found in Appendix 5.

Animal Body Weights

Animal body weights were determined (in kg) with a certified, verified scale on study Days −1 and 21. The recorded weights were used to determine individual treatment doses.

Anaesthesia and Sodium Urate Injection

Induction of synovitis by sodium urate injection occurred on Days 0, 2, 7 and 22, as outlined in the Schedule of Operations, and employed the following procedures for each subject. Anaesthesia was induced with propofol (8 mg/kg IV to effect). Subjects were then intubated and anaesthesia was maintained with an isoflurane-oxygen mixture. If IV administration of the propofol was not possible due to inaccessibility of the vein, the subjects were masked down using the isoflurane-oxygen mixture and the event was recorded on the anaesthesia record.

Prior to injection, sodium urate crystals were mixed with sterile saline to produce a solution with a concentration of 20 mg/ml. The solution was sonicated for 60 minutes and the pH was adjusted to a suitable level for injection (between 6.9 and 7.2) with the addition of either hydrochloric acid or sodium hydroxide.

Following aseptic surgical preparation of the injection area, the joint was aspirated and the presence of synovial fluid confirmed accurate location for injection. For each animal, 1 ml of the sodium urate solution was injected into either the right or left stifle. For the second urate injection (if applicable), the stifle contralateral to that used for the first urate injection was used. Each treatment group had 4 dogs induced in the left stifle, and 4 dogs induced in the right stifle.

Pain Questionnaire Assessment

The pain questionnaire is a laboratory adaption of the validated canine brief pain inventory (CBPI) questionnaire, which is a clinical questionnaire used to evaluate pain level based on owner responses. Modifications to the questionnaire account for differences between owner pain evaluation of pets and pain evaluation of laboratory dogs by technical staff. Specifically, the ability of each dog to walk, trot, gallop, rear, jump over a low obstacle, climb and descend stairs, jump down from a perch and general activity is evaluated in parallel to subjective evaluation of pain observed during each behavior. Assessments were performed by the same trained technician across the study.

All subjects underwent pain questionnaire pre-screening within 3 days of each scheduled induction as outlined in the Schedule of Operations. Cumulative pain scores were used to confirm inclusion criteria and also served as the baseline scores for the treatment administered. Subjects assigned to more than one treatment group were re-screened prior to the second induction. This served two purposes; first to ensure animals returned to normal pain levels following the initial sodium urate injection, and second, to ensure that subject's cumulative pain scores continued to be below 5 as per the inclusion criteria for the study.

On days sodium urate was injected, animals were assessed on the questionnaire at 4.5, 8.5, 24.5 and 30.5 hours following induction of synovitis (±15 minutes). At these time points, the pain questionnaire was performed immediately following measurement of the patella/patellar tendon (knee cap) width as described below.

Caliper Measurements

Within thirty minutes of synovitis induction and 4.5, 8.5, 24.5 and 30.5 hours following induction (±15 minutes), the width of the patella and the patellar tendon were measured in millimeters using Venier 150 mm plastic gauge calipers which measured in increments of 1 mm. Measurements of both the left and right stifle were performed by the same technician, who took three separate caliper measurements of each joint. The three measurements from each joint were averaged. On the day prior to the pre-induction caliper measurement, the hair of the measurement areas were clipped and marked to facilitate consistent measures.

Blinding of the Study

The treatment given to each animal was not revealed to technicians collecting the pain and caliper data. The study was blinded to all personnel in the investigation with the exception of the persons involved with preparing and administering the investigational products, the person responsible for performing the allocation, the Study Coordinator and Scientific Director. Non-blinded personnel did not collect data other than at the time of treatment. Treatment condition information was kept in a locked archive room over the course of the study.

Statistical Analysis

Mean+/−standard error of mean for pain scores, function scores, and knee measurements were compared by T test. Only statistically significant (p<0.05) are indicated in the results below.

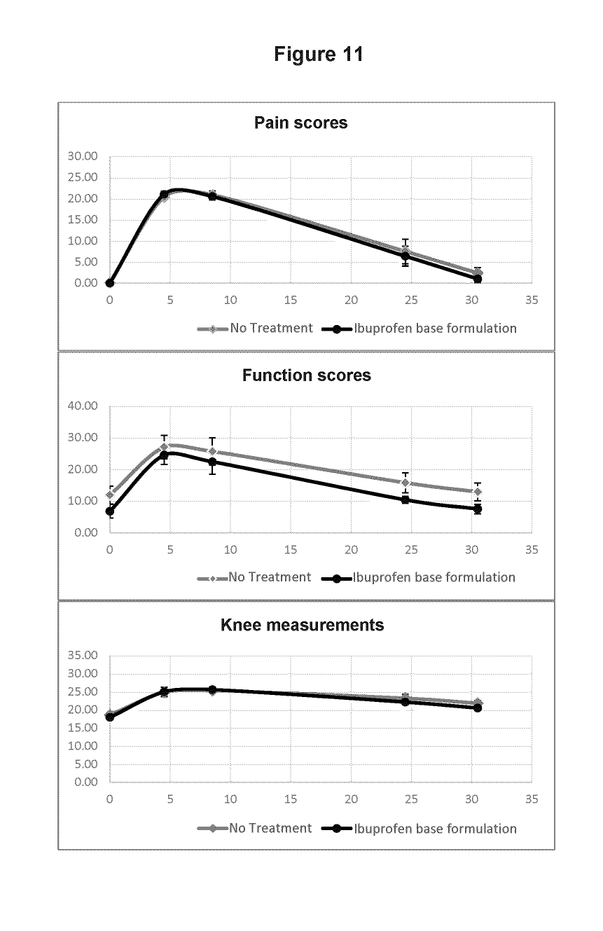

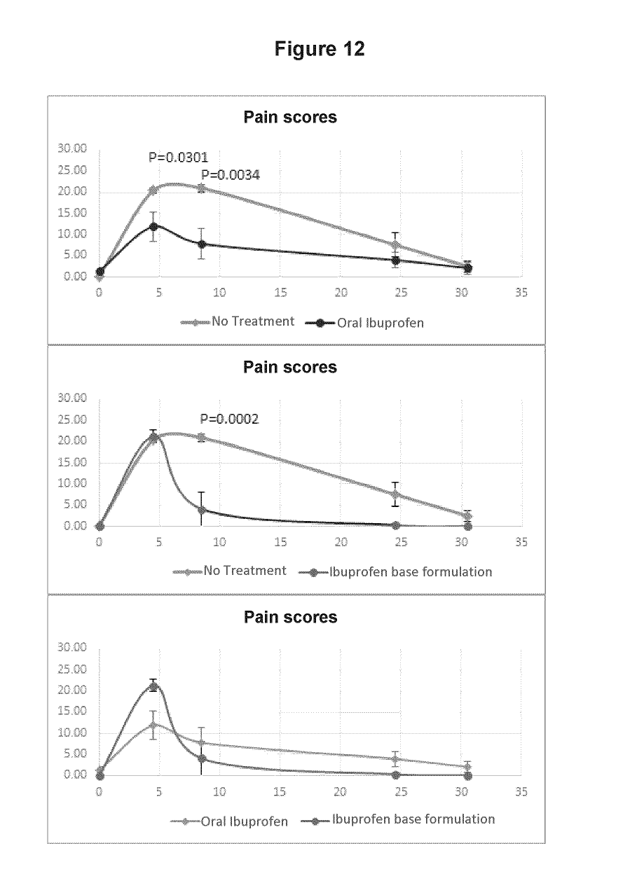

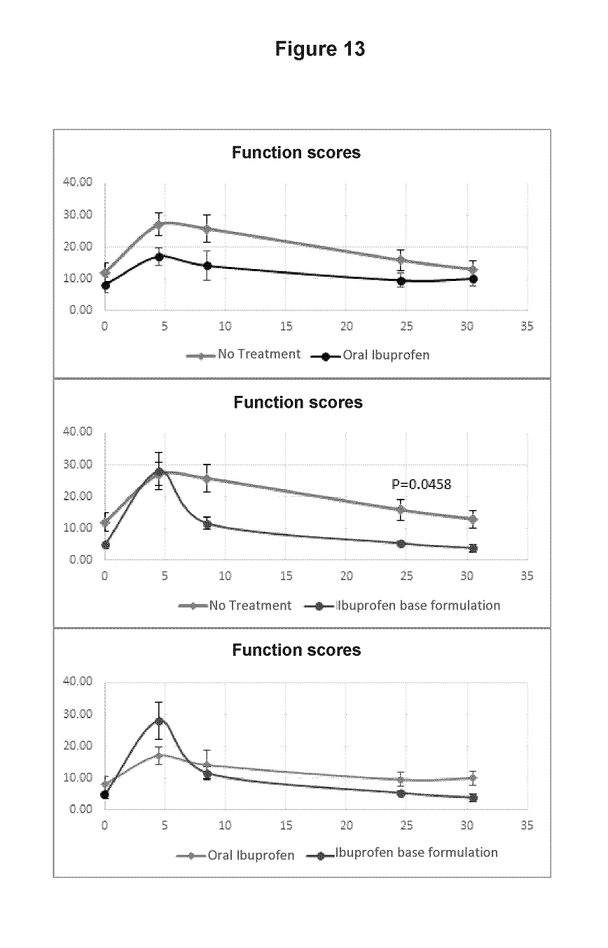

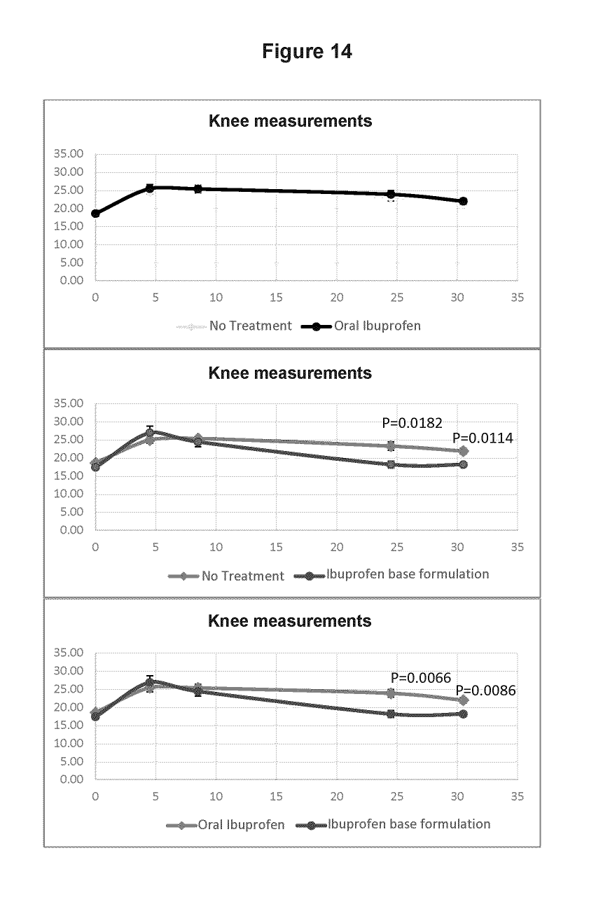

The results of this study are shown in FIGS. 11-14. FIG. 11 shows that a transdermal base formulation of the disclosure does not affect pain scores (upper panel), function scores (middle panel), or knee measurements (bottom panel) when compared to untreated animals. FIG. 12 shows the pain scores for ibuprofen treatment by oral administration (PO) (upper panel) or transdermal base formulation of the disclosure (4). Direct comparison of oral vs base formulation of Ibuprofen revealed no statistically significant differences in pain scores (lower panel). FIG. 13 shows joint function scores for ibuprofen treatment by oral administration (upper panel) or transdermal base formulation of the disclosure (middle panel). Direct comparison of oral vs base formulation formulations of Ibuprofen revealed no statistically significant differences in function scores (lower panel). FIG. 14 shows knee caliper measurements for ibuprofen treatment by oral administration (upper panel) or transdermal base formulation of the disclosure (middle panel). Direct comparison of oral vs base formulations of Ibuprofen revealed a statistically improvement in knee measurement scores (lower panel).

The results show that the base formulation alone has no effect on pain, function, or knee measurement when compared with untreated animals suggesting that any effect observed in this series of experiments is due to the Ibuprofen and not the base. Transdermal delivery of Ibuprofen requires a longer period to affect a change in pain, however longterm pain changes are equivalent regardless of administration route. Transdermal delivery of Ibuprofen allowed more rapid restoration of function in the knee joint. Transdermal delivery of Ibuprofen reduced the swelling of the knee joint as compared to oral Ibuprofen. Transdermal Ibuprofen outperforms oral Ibuprofen for long term reduction in swelling of knee joint.

### Example 8: Oral and Transdermal Ibuprofen in Canine Sera and Synovial Fluid

The objective of this study was to determine if the transdermal base of the disclosure is capable of delivering ibuprofen into the systemic circulation and joint synovial fluid as well as to compare transdermal versus oral routes of administration.

Methods

Ibuprofen:

12 dogs with radiographic evidence of osteoarthritis (OA) were utilized to compare the concentration of ibuprofen in both blood and synovial fluid following transdermal delivery of ibuprofen formulated in base formulation and oral delivery in capsules. The 12 dogs were randomly divided into two groups of six subjects; one group received the oral positive treatment control and the other group received the transdermal treatment. The treatment cycle consisted of six days twice daily followed by a period of seven days devoid of treatment. Blood and synovial fluid samples (from both stifles and shoulders) were collected on the last (sixth) day of drug application and on the last (seventh) day of the washout period.

Test article: 1.5% (w/w) ibuprofen in base formulation (4); ˜1 g gel per shoulder joint twice daily (i.e. 15 mg ibuprofen per shoulder joint twice daily). Control article: base formulation; ˜1 g gel per shoulder joint twice daily. Positive Control: 30 mg ibuprofen in capsule PO twice daily.

Serum and Synovial Fluid Collection:

Whole blood and synovial fluid samples were collected from the dogs 60 mins post treatment on the 7^{th} day, and additional samples were collected after a 7 day washout. Baseline synovial fluid was collected from untreated canine subjects. Synovial fluid was collected under sedation (medetomidine (0.01 mg/kg) and butorphenol (0.1 mg/kg) IV). The fluid was collected by arthrocentesis into the joint following surgical preparation of the area. Synovial fluid was collected from stifle or shoulder joints.

Analytical Method Development and Quantification:

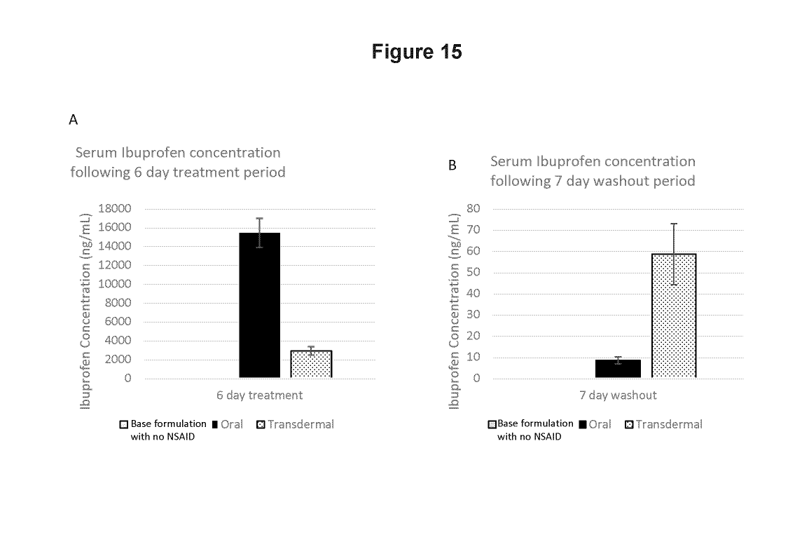

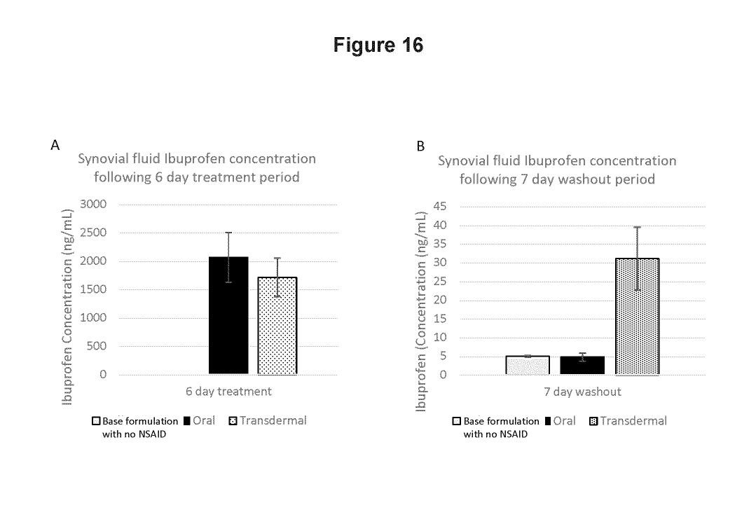

Following submission of raw data, the results were categorized and summarized for average and deviations for each treatment group and time period as shown in Table 11 (Table A-C: Outline of Experimental Design and Drug Concentrations). The table depicts the drug concentrations used during the chronic administration period of six days (A), the total number of animals with biological samples (analyzed in duplicate) for each drug treatment and time period (B), and the final drug concentrations (analyzed in duplicate) for each drug treatment and time period (C) (regardless of route of administration, ibuprofen was given at a rate of 30 mg/day). Drug concentrations (C) are described and include values for the standard deviations for each in brackets. Statistically significant differences between oral and transdermal drug concentrations are highlighted (*, Pvalue=0.00002; ^{†}Pvalue=0.02). FIGS. 15 and 16 depict the data in the table. FIG. 15 shows Chronic (6 day) administration of oral or transdermal ibuprofen (A) yields a serum drug concentration of 15.4 μg/mL and 2.9 μg/mL, respectively (*p value=0.00002). This data indicates that microgram levels of ibuprofen within the circulatory system can be achieved using transdermal applications, however the resulting concentration is approximately 5-fold lower compared to an oral regimen. Following a seven day period devoid of drug administration the levels of ibuprofen in oral or transdermal treatments (B) decreased to 8.5 ng/mL and 58.5 ng/mL, respectively. This data indicates that a one week washout period is sufficient to reduce circulating drugs by >95%. The observation that transdermal levels are higher than oral following washout (^{†}p value=0.02) indicates a slow desorption of the drug from the application site. Levels observed in serum are equivalent to those observed in plasma samples (data not shown). Error bars depicted standard error of the mean.

FIG. 16 shows Ibuprofen concentration in synovial fluid. Chronic (6 day) administration of oral or transdermal ibuprofen (A) yields a synovial fluid drug concentration of 2065 ng/mL and 1711 ng/mL, respectively. The observed oral and transdermal-derived drug levels in synovial fluid are not statistically different (p value=0.86). This data indicates equivalent synovial fluid levels are achieved regardless of route of administration. Following a seven day period devoid of drug administration the levels of ibuprofen in oral or transdermal treatments (B) decreased to 8.5 ng/mL and 58.5 ng/mL, respectively. This data indicates that a one week washout period is sufficient to reduce circulating drugs by >95%. The observation that transdermal levels are higher than oral (^{†}p value=0.02) following washout indicates a slow desorption of the drug from the application site. Error bars depicted standard error of the mean.

The results of this experiment include several observations regarding the relative bioaccumulation of ibuprofen in the systemic circulation when comparing oral versus transdermal routes of administration. With respect to ibuprofen, when canines are exposed to equimolar amounts by oral or transdermal routes the relative bioavailability of the transdermal is ˜fivefold lower in the systemic circulation. In contrast, the levels of ibuprofen within the joint synovial fluid are approximately equivalent regardless of the route of administration. This observed equivalence in synovial fluid and the evident difference in circulating levels is indicative of an altered tissue distribution profile for transdermal ibuprofen. Lastly, measurable levels of ibuprofen in transdermally treated animals following the seven day washout period, whereas the orally treated animals were essentially devoid of drug. This indicated transdermal base formulation-ibuprofen provides a longer lasting and sustained release of drug within the target tissues.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

TABLE 1
Formulation 1

| | Ingredients | % |
|---|---|---|
| Phase A | Emulsifier | 4.0% |
| | Wax Stabilizer | 5.0% |
| | Polar Emollient Oils | 10% |
| | Medium Polar Emollient | 2.0% |
| Phase B | Water | 55.70% |
| | Humectant | 4% |
| | Thickening agent | 0.50% |
| Phase C | Preservatives | 1.3% |
| Phase D | Flavonoid-containing extracts | 4% |
| | Thickening Agent | 1.0% |
| Phase E | Water | 4.0% |
| | Phospholipid-Complexed Flavonoid | 2.0% |
| Phase F | Penetration Enhancer | 5.0% |
| | Naproxen | 1.5% |
| | Total | 100.00% |

TABLE 2
Formulation 2

| | Ingredients | % |
|---|---|---|
| Phase A | Emulsifier | 4.0% |
| | Wax Stabilizer | 4.0% |
| | Polar Emollient Oils | 10% |
| | Medium Polar Emollient | 2.0% |
| Phase B | Water | 48.20% |
| | Humectant | 4% |
| | Thickening agent | 0.20% |
| Phase C | Preservatives | 1.3% |
| Phase D | Flavonoid-containing extracts | 6% |
| | Thickening Agent | 1.0% |
| Phase E | Water | 3.14% |
| | Phospholipid-Complexed Flavonoid | 2.0% |
| Phase F | Diclofenac Sodium | 1.16% |
| | Penetration Enhancer | 12.0% |
| | Emulsifier | 1.0% |
| | Total | 100.00% |

TABLE 3
Stability parameters, pH, texture, color and
odour for formulation 2 at 45° C. for 90 days

| | Avg. | | Viscosity | | | |
|---|---|---|---|---|---|---|
| | Temp | | (T4-0.6 rpm, | Appear- | | |
| Months | (° C.) | pH | cps) | ance | Colour | Scent |
| 0 | 25 | 7.02 | 435000 | cream | yellow | characteristic |
| 1 | 25 | 6.45 | 429000 | cream | yellow | characteristic |
| 2 | 25 | 6.67 | 434000 | cream | yellow | characteristic |
| 3 | 25 | 6.39 | 329000 | cream | yellow- | characteristic |
| | | | | | brown | |

TABLE 4
Formulation 3

| | Ingredients | % |
|---|---|---|
| Phase A | Emulsifier | 4.0% |
| | Wax Stabilizer | 5.0% |
| | Polar Emollient Oils | 10% |
| | Medium Polar Emollient | 3.0% |
| Phase B | Water | 48.70% |
| | Humectant | 4.0% |
| | Thickening agent | 0.50% |
| Phase C | Preservatives | 1.3% |
| Phase D | Flavonoid-containing extracts | 7% |
| | Thickening Agent | 1.0% |
| Phase E | Water | 2.0% |
| | Phospholipid-Complexed Flavonoid | 2.0% |
| Phase F | Ibuprofen | 4.0% |
| | Penetration Enhancer | 7.5% |
| | Total | 100.00% |

TABLE 5
The stability parameters, pH, texture, color and
odour for formulation 3 at 45° C. for 90 days

| | Avg. | | Viscosity | | | |
|---|---|---|---|---|---|---|
| | Temp | | (T4-1.5 rpm, | Appear- | | |
| Months | (° C.) | pH | cps) | ance | Colour | Scent |
| 0 | 25 | 5.93 | 160400 | cream | beige - | characteristic |
| | | | | | yellow | |
| 1 | 25 | 5.60 | 354800 | cream + | beige - | characteristic |
| | | | | drops oil | yellow | |
| 2 | 25 | 5.80 | 326800 | cream + | beige - | characteristic |
| | | | | drops oil | yellow | |
| 3 | 25 | 5.57 | 216800 | cream + | beige - | characteristic |
| | | | | drops oil | yellow | |

TABLE 6
Formulation 4

| | Ingredients | % |
|---|---|---|
| Phase A | Emulsifier | 4.0% |
| | Wax Stabilizer | 5.0% |
| | Polar Emollient Oils | 10% |
| | Medium Polar Emollient | 3.0% |
| Phase B | Water | 30.20% |
| | Humectant | 4.0% |
| | Thickening agent | 0.50% |
| Phase C | Preservatives | 1.3% |
| Phase D | Flavonoid-containing extracts | 7% |
| | Thickening Agent | 1.0% |
| Phase E | Water | 2.0% |
| | Phospholipid-Complexed Flavonoid | 2.0% |
| Phase F | Ketoprofen | 15.0% |
| | Penetration Enhancer | 15.0% |
| | Total | 100.00% |

TABLE 7
The stability parameters, pH, texture, color and
odour for formulation 4 at 45° C. for 90 days

| | Avg. | | Viscosity | | | |
|---|---|---|---|---|---|---|
| | Temp | | (T4-5.0 rpm, | Appear- | | |
| Months | (° C.) | pH | cps) | ance | Colour | Scent |
| 0 | 25 | 4.57 | 55100 | cream | beige - | characteristic |
| | | | | | yellow | |
| 1 | 25 | 4.61 | 11300 | cream | beige - | characteristic |
| | | | | | yellow | |
| 2 | 25 | 4.64 | 8300 | cream + | beige - | characteristic |
| | | | | drops oil | yellow | |
| 3 | 25 | 4.44 | 5200 | cream + | beige - | characteristic |
| | | | | oil | yellow | |

TABLE 8
Formulation 5

| | Ingredients | % |
|---|---|---|
| Phase A | Emulsifiers | 7% |
| | Wax Stabilizer | 2.5% |
| | Polar Emollient Oils | 11.5% |
| | Medium Polar Emollient | 4.5% |
| Phase B | Water | 53.55% |
| | Humectant | 3% |
| | Thickener | 0.4% |
| | Chelating Agent | 0.05% |
| Phase C | Flavonoid Containing Extracts | 7% |
| Phase D | Preservative | 1.1% |
| | Solubilizer | 0.5% |
| | Penetration Enhancer | 1.5% |
| Phase E | Phospholipid-complexed Rutin | 2% |
| | Water | 2% |
| Phase F | Buffering Agents | 1% |
| | Water soluble preservative booster | 0.8 |
| | Water | 1.6% |
| | Total | 100.00% |

TABLE 9
Formulation 6

| | Ingredients | % |
|---|---|---|
| Phase A | Formulation 5 | 70% |
| | Ketoprofen | 15% |
| | Penetration Enhancer | 15% |
| | Total | 100.00% |

TABLE 10

| | | | | Outcome/ | |
|---|---|---|---|---|---|
| | | | | Inflam- | Outcome/ |
| Gender | Age | Symptoms | % Cream* | mation | Pain |
| Female | 72 | Inflamed hands | 1.5%* | + | + |
| | | with pain, low | Naproxen | | |
| | | mobility | | | |
| Female | 74 | Pain/inflam- | 1.5% | + | + |
| | | mation in both | Naproxen | | |
| | | knees | | | |
| Male | 67 | Pain/inflam- | 1.5% | + | + |
| | | mation in hands | Naproxen | | |
| | | & shoulders | | | |
| Female | 79 | Pain/inflam- | 1.5% | + | Short- |
| | | mation in neck | Naproxen | | lived, mild |
| | | and higher back | | | relief |
| Male | 55 | Pain/inflam- | 15%* | + | + |
| | | mation in hands | Ketoprofen | | |
| Male | 52 | Chronic | 15% | + | + |
| | | pain/inflam- | Ketoprofen | | |
| | | mation on right | | | |
| | | knee | | | |
| Male | 80 | Chronic | 15% | Little | + |
| | | pain/inflam- | Ketoprofen | change in | |
| | | mation entire | | swelling | |
| | | back | | | |
| Male | 53 | Chronic knee | 15% | + | + |
| | | pain/inflam- | Ketoprofen | | |
| | | mation | | | |
| Female | 60 | Inflamed ankle/ | 15% | + | + |
| | | pain | Ketoprofen | | |
| Male | 45 | Pain with hip | 15% | No | + |
| | | | Ketoprofen | change in | |
| | | | | swelling | |
| Male | 86 | Chronic shoulder | 15% | No | + |
| | | pain | Ketoprofen | swelling | |
| Male | 50 | Chronic ankle | 15% | + | + |
| | | pain | Ketoprofen | | |
| Female | 49 | Chronic facial | 15% | + | + |
| | | pain/trigeminal | Ketoprofen | | |
| | | neuralgia | | | |

*1.5% naproxen = formulation 1
15% ketoprofen = formulation 4

TABLE 11 A-C
Outline of Experimental Design and Drug Concentrations

A
Chronic Administration (6 days)

| | Oral | Transdermal |
|---|---|---|
| | (mg/day) | (mg/day) |
| Ibuprofen | 30 | 30 |

| | B | | | |
|---|---|---|---|---|
| | Chronic Administration (6 days) | | Washout (7 days) | |
| | Oral | Transdermal | Oral | Transdermal |
| | (N) | (N) | (N) | (N) |
| Ibuprofen | | | | |
| Serum | 5 | 6 | 5 | 6 |
| Synovial | 6 | 6 | 6 | 6 |
| Fluid | | | | |

| | C | | | |
|---|---|---|---|---|
| | Chronic Administration (6 days) | | Washout (7 days) | |
| | Oral | Transdermal | Oral | Transdermal |
| | (ng/mL) | (ng/mL) | (ng/mL) | (ng/mL) |
| Ibuprofen | | | | |
| Serum | 15,420 (±3,450)* | 2,904 (±1,212)* | 9 (±4)^{□} | 59 (±38)^{□} |
| Synovial | 2,065 (±1,070) | 1,711 (±828) | 5 (±3)^{□} | 31 (±21)^{□} |
| Fluid | | | | |

### REFERENCES

1. Sharma, S., Prasad, A., Anand, K. S. (1999). Nonsteroidal anti-inflammatory drugs in the management of pain and inflammation: a basis for drug section. Am. J. Therap. 6: 3-11.
2. Canadian agency for drugs and technologies in health. (2013). Non-steroidal anti-inflammatory drugs for pain: a review of safety.
3. Botting, R. M. (2006). Inhibitors of cyclooxygenases: mechanisms, selectivity and uses. J. Physiol. Pharmacol. 57: 113-124.
4. Bushra, R., Aslam, N. (2010). An overview of clinical pharmacology of ibuprofen. Oman. Med. J. 25: 155-161.
5. Brogden, R. N. (1986). Non-steroidal anti-inflammatory analgesics other than salicylates. Drugs. 4:27-45.
6. Prausnitz, M. R., Langer, R. (2008). Transdermal drug delivery. Nat.

Biotechnol. 26:1261-1268.
7. Conaghan, P. G. (2012). A turbulent decade for NSAIDs: update on current concepts of classification, epidemiology, comparative efficacy, and toxicity. 32:1491-1502.
8. Yiyun, C., Na, M., Tongwen, X., Rongqiang, F., Xueyuan, W., Xiaomin, W., Longping, W. (2007). Transdermal delivery of nonsteroidal anti-inflammatory drugs mediated by polyamidoamine (PAMAM) dendrimers. J. Pharm. Sci. 96:595-602.

1. A transdermal formulation comprising,
(a) an aqueous phase comprising water and at least one water soluble emulsion stabilizer;
(b) an oil phase comprising at least one emulsifier, at least one oil soluble emulsion stabilizer, at least one emollient comprising at least one flavonoid and at least one other emollient;
wherein the oil and aqueous phase form an emulsion;
(c) an external phase comprising at least one flavonoid containing-extract, at least one phospholipid-complexed flavonoid and at least one NSAID selected from a propionic acid NSAID and an acetic acid NSAID; and optionally
(d) at least one preservative phase.

2. The transdermal formulation of claim 1, wherein the propionic acid NSAID is selected from one or more of ibuprofen, naproxen, fenoprofen, ketoprofen, flurbiprofen, oxaprozin and loxoprofen.

3. The transdermal formulation of claim 2, wherein the propionic acid NSAID is ibuprofen, naproxen or ketoprofen.

6. The transdermal formulation of claim 1, wherein the propionic acid NSAID is present in the formulation in an amount of about 0.5 wt % to about 25 wt %, about 1 wt % to about 20 wt %, or about 1.5 wt % to about 15 wt % of the total formulation.

7. The transdermal formulation of claim 1, wherein the acetic acid NSAID is selected from one or more of indomethacin, tolmetin, sulindac, etodolac, diclofenac, aceclofenac and nabumetone.

8. The transdermal formulation of claim 7, wherein the acetic acid NSAID is diclofenac or diclofenac sodium.

10. The transdermal formulation of claim 7, wherein the acetic acid NSAID is present in the formulation in an amount of about 0.5 wt % to about 5 wt %, or about 1 wt % to about 3 wt % of the total formulation.

11. The transdermal formulation of claim 1 in the form of a cream, gel, liquid suspension, ointment, solution or patch.

12. The transdermal formulation of claim 1, in the form of a cream.

13. The transdermal formulation of claim 12, wherein the cream has a viscosity of about 10000 cps to about 500000 cps, or about 15000 cps to about 450000 cps as measured using a Brookfield RVT T4-0.5, T4-0.6 or T4-1.5 RPM instrument at room temperature.

14. The transdermal formulation of claim 1, wherein the formulation is a sustained release formulation.

15. A method for the transdermal administration of one or more NSAID selected from a propionic acid NSAID and an acetic acid NSAID comprising administering an effective amount of one or more of the formulations of claim 1 to a subject in need thereof.

16. A method for treating a NSAID-responsive disease or condition comprising administering an effective amount of one or more of the transdermal formulations of claim 1 to a subject in need thereof.

17. The method of claim 15, wherein the NSAID-responsive disease or condition is selected from one or more of acute pain, chronic pain, nociceptive pain, neuropathic pain, inflammation, migraine, cancer, ankylosing spondylitis, heart disease, neurodegenerative disease and auto-immune disease.

18. The method of claim 16, wherein the acute pain is selected from musculoskeletal pain, postoperative pain and surgical pain.

19. The method of claim 16, wherein the chronic pain is selected from rheumatoid arthritis, osteoarthritis, pain associated with cancer and fibromyalgia.

20. The method of claim 16, wherein the cancer is selected from breast, colorectal, prostate and non-small cell lung.

21. The method of claim 16, wherein the auto-immune disease is selected from multiple sclerosis and lupus.

22. A method for the sustained release of one or more NSAID selected from a propionic acid NSAID and an acetic acid NSAID comprising administering an effective amount of one or more of the formulations of claim 1 to a subject in need thereof.
